(19) United States
(12) Patent Application Publication
(10) Pub. No.: US 2022/0096834 A1
(43) Pub. Date: Mar. 31, 2022

(54) Methods and Devices for Improving Sensory Perception by Tonic Vagus Nerve Stimulation

(71) Applicant: The Trustees of Columbia University in the City of New York, New York, NY (US)

(72) Inventors: Qi WANG, New York, NY (US); Charles RODENKIRCH, New York, NY (US)

(73) Assignee: The Trustees of Columbia University in the City of New York, New York, NY (US)

(21) Appl. No.: 17/547,436

(22) Filed: Dec. 10, 2021

Related U.S. Application Data
(63) Continuation of application No. PCT/US2020/037660, filed on Jun. 14, 2020.
(60) Provisional application No. 62/861,715, filed on Jun. 14, 2019.

(51) Int. Cl. A61N 1/36

(52) U.S. Cl. CPC A61N 1/36025; A61N 1/36031; A61N 1/36034

(57) ABSTRACT

Methods and devices for modifying sensory processing in a subject are provided. Aspects are directed to applying tonic vagus nerve stimulation to a subject for transient sensory processing modification. Devices for applying tonic vagus nerve stimulation when a subject is in need of sensory modification or on demand are also provided. The devices can be coupled with a prosthetic device for application to regions of the body in need of vagus nerve stimulation.

All references cited herein, including, but not limited to patents and patent applications, are incorporated by reference in their entirety. This application is a Continuation of International Application No. PCT/US2020/037660, filed on Jun. 14, 2020, which claims priority to and the benefit of U.S. Provisional Patent Application No. 62/861,715 filed Jun. 14, 2019, each of which is hereby incorporated by reference in its entirety.

### STATEMENT REGARDING FEDERALLY SPONSORED RESEARCH OR DEVELOPMENT

This invention was made with government support under 1847315 awarded by the National Science Foundation and MH112267 awarded by the National Institutes of Health. The government has certain rights in the invention.

### BACKGROUND

Recent work has shown that the locus coeruleus (LC), the sole source of norepinephrine (NE) to the forebrain, provides behavioral-state-relevant modulation of the neural coding in the early stage of the somatosensory pathway¹. Specifically, it was found that LC activation enhances thalamic feature selectivity via norepinephrine regulation of intrathalamic circuit dynamics. Modulation of sensory processing has many translational applications; however, the LC is a deep brainstem nucleus which prevents direct noninvasive activation with currently available techniques²⁻⁴. However, peripheral nerve stimulation techniques provide a pathway for treatment, due to their ability to readily activate downstream neuromodulatory systems with minimal invasiveness and reduced side effects⁵. Previous research has shown that vagus nerve stimulation (VNS) activates the LC⁶. Further, VNS has been approved by the FDA (U.S. Food and Drug Administration) for use in treatment of epilepsy and tinnitus in humans, and has been proposed as a treatment for a wide variety of neurodisorders including depression, autism, stroke-induced damage, and PTSD (post-traumatic stress disorder)⁷⁻¹³. Recently, techniques allowing for non-invasive transcutaneous VNS have been developed and commercially implemented¹⁴⁻¹⁷. VNS has been shown to activate neuromodulatory networks, including the locus-coeruleus-norepinephrine (LC-NE) system^{6,18}.

Previous work has focused on using the VNS to facilitate the neuroplasticity of brain circuits, likely through activation of neuromodulatory pathways¹⁹. These VNS-induced neuroplasticity-driven changes can persist over long timescales²⁰.

Locus Coeruleus (LC) activation improves feature selectivity in the ventral posteromedial nucleus (VPm), effectively increasing the sensory-stimulus related information transmitted by thalamic relay neurons to the cortex resulting in improved perception of details of sensory stimuli¹. Vagus nerve stimulation (VNS) can be used to increase LC activity⁶. VNS has been studied as a therapy to treat neurological disorders including epilepsy, depression, stroke, and tinnitus. LC activation has been correlated with pupil diameter²¹.

When sensory information enters the brain, it is encoded as a neural signal. The encoded sensory information is then processed through multiple brain regions prior to perception. This processing of sensory information is imperfect, introducing noise that degrades the accuracy of the resulting perception. Therefore, perceptual acuity is dependent on the quality of sensory processing.

Accurate perception of details in tactile, auditory, and visual stimuli is useful for performing tasks correctly and safely. Once sensory information is encoded as neural activity, it is processed through multiple brain regions (i.e. thalamus, cortex) before perception occurs. Therefore, perceptual acuity is dependent upon high-fidelity, accurate processing of sensory stimuli by the brain (i.e. sensory processing). Accuracy of perception exerts a heavy influence on an individual's ability to complete workplace tasks, compete at sports, or even enjoy hobbies. Unfortunately, sensory loss is all too common. For example, one study found 94 percent of adults over 57 years of age had a deficiency in at least one sensory modality²². This suggests that, in the United States, roughly 64 million suffer from some form of age-related sensory loss. As the elderly population grows, the population suffering from age-related sensory loss will increase, stressing current facilities that are not well designed to accommodate individuals with impaired senses²³. However, elderly individuals are not the only ones at risk of sensory loss. In addition to aging, traumatic brain injury (TBI) and various neurological disorders can also degrade sensory acuity²⁴⁻²⁶. Finally, even individuals with normally accurate perception can occasionally suffer from impaired senses. This is because there are multiple commonly occurring factors, such as fatigue and inattention^{27,28}, that can degrade the sensory acuity of individuals with usually healthy senses.

Our reliance on our senses makes sensory loss highly disruptive to quality of life. Sensory loss is well-known to be isolating and can have devasting effects on mental health²⁹. Impaired senses in the elderly are especially damaging as they can interfere with their ability to live independently. For example, compromised sense of touch often leads to difficulty buttoning shirts³⁰ or grasping objects needed to complete personal hygiene tasks. Degraded visual and auditory senses result in communication breakdown²³ and stress important support relationships¹. The combined effects of sensory loss often result in depression, anxiety, and withdrawal from social situations. Finally, sensory loss is associated with increased risk of accidents, such as falls, that can have life threatening consequences³¹. Even temporarily impaired senses in otherwise healthy individuals, which can occur due to fatigue or inattention^{27,28}, can cause significant negative effects. For example, sensory misperceptions arising from degraded sensory acuity can result in costly human error for military service members or workers who operate heavy machinery. Further, for individuals competing at sports or e-sports where peak performance is key, inaccurate perception can cause incorrect decisions and failure.

There is currently a dearth of available methods for improving sensory processing and those that do exist have many drawbacks. Stimulants improve sensory processing but cause cardiac damage³², insomnia, anxiety, and addiction^{33,34}. Various nootropics brands make often unverified claims their supplements improve brain function. However, nootropics are largely ineffective and occasionally dangerous due lack of proper testing³⁵. For example, one research group found that after they published minimal preclinical research suggesting a compound might improve cognitive function, a nootropics company begun marketing the compound without any tests of long-term toxicity³⁶. Consumers' willingness to potentially risk their health by consuming research grade compounds without clinical testing highlights an unfulfilled need for technology that can improve sensory ability. Finally, as both stimulants and nootropics are taken orally, their effect has a delayed onset (30 to 60 minutes from ingestion) and cannot be turned off if desired. Taken together, these observations make it clear there is an unmet clinical need for bioelectronic technology that can improve sensory processing on-demand without risk of addiction, cardiac damage, or insomnia.

What is needed are methods and devices to improve perception of sensory information to, for example, enhance sensory perception and treat sensory components of neurological disorders.

### SUMMARY OF THE INVENTION

Aspects described herein provide methods of modifying sensory processing in a subject by applying a tonic vagus nerve stimulation to the subject wherein the sensory processing of the subject is modified. The rapid, and transient effects of VNS can substantially affect the sensory processing within the thalamus on a short timescale. This new application of VNS does not depend on long-term changes induced by neuroplasticity, but rather utilizes VNS for short-term, rapid improvement of sensory processing in the thalamus (e.g., effects disappear within a minute of cessation of VNS).

In another aspect, tonic VNS (e.g., extended tonic VNS) can improve thalamic sensory processing through increasing the feature selectivity and information transmission efficiency and rate of sensory neurons. As described herein, traditional duty-cycled VNS is sub-optimal for sensory enhancement as it creates a fluctuating bias on sensory evoked response due to the rapid, transient nature of the effects of VNS on sensory processing. Methods and apparatus described herein use VNS to improve behavioral performance in perceptual tasks.

Further aspects provide methods of modifying sensory processing in a subject, by determining a mean value and a variance value for the pupil diameter from the first time point to the second time point; measuring the pupil diameter and determining a pupil diameter value; and applying tonic vagus nerve stimulation to the subject when the pupil diameter value is at least about one to three standard deviations from the variance value for pupil diameter. In some instances, the subject is exposed to a sensory stimulation.

In yet another aspect, methods of modifying sensory processing in a subject by exposing the subject to a sensory stimulation; measuring a change (e.g., sampling a measurement over the time range from a first time point to a second time point) in a pupil diameter from a first time point to a second time point; determining a mean value and a variance value for the pupil diameter from the first time point to the second time point; measuring the pupil diameter and determining a pupil diameter value; and applying tonic, continuous vagus nerve stimulation to the subject when the pupil diameter value is at least about one to three standard deviations from the variance value for pupil diameter are provided.

Aspects described herein provide methods of modifying sensory processing in a subject by detecting when the subject is in need of a sensory processing modification; applying tonic vagus nerve stimulation to the subject to provide the sensory processing modification; and discontinuing applying the sensory processing modification when the subject no longer is in need of sensory processing modification.

Further aspects provide a method of modifying sensory processing in a subject, by measuring a change (e.g., sampling a measurement over the time range from a first time point to a second time point) in a bioelectronic signal (e.g., EEG (synchronization, relative power band strength, spatial pattern analysis),), EKG (heart rate, heart rate variability), change in blood pressure, ECOG (synchronization, relative power band strength, spatial pattern analysis), respiratory rate, perspiration (e.g., measured by conductivity of skin surface), or a signal recorded from invasive or noninvasive brain-machine interface) from a first time to a second time; determining a mean value and a variance value for the signal from the first time to the second time; measuring the bioelectronic signal and determining a measured value for the bioelectronic signal; and applying tonic vagus nerve stimulation to the subject when the measured value is at least about one to three standard deviations from the variance value.

Further aspects provide vagus nerve stimulation devices adapted to apply tonic vagus nerve stimulation to a subject to modify sensory processing in the subject, wherein a time of applying the tonic vagus nerve stimulation for at least about 3 seconds, at least about 30 seconds, or at least about 4 minutes.

### BRIEF DESCRIPTION OF THE DRAWINGS

FIG. 1A shows an exemplary diagram of an experimental setup and VNS electrode cuff implantation;

FIG. 1B shows an exemplary VPm neuron response to punctate stimulation of the animal's principle whisker;

FIG. 1C shows exemplary whisker and VNS patterns;

FIG. 1D shows an exemplary summary of feature modulation factor during the control period versus the end of the rest period;

FIG. 1E shows an exemplary summary of the percent of spikes in bursts during the control period versus the end of the rest period;

FIG. 1F shows an exemplary summary of improvement in information transmission efficiency during the control period versus the end of the rest period;

FIG. 2A shows an exemplary spike raster plot of an example VPm response to repeated presentation of the same white Gaussian noise (WGN) whisker stimulation referenced above with and without VNS;

FIG. 2B shows an exemplary firing rate of VPm neurons to the same WGN whisker stimulation referenced above with and without VNS;

FIG. 2C shows an exemplary linear-nonlinear Poisson model used for white noise reverse correlation analysis;

FIG. 2D shows an exemplary kinetic feature encoded by an example VPm neuron recovered with and without VNS and corresponding nonlinear tuning functions (inset);

FIG. 2E shows an exemplary summary of feature modulation factor with and without VNS;

FIG. 2F shows an exemplary summary of improvement in information transmission efficiency by VNS;

FIG. 2G shows an exemplary summary plot of information conveyed by tonic spikes, burst spikes, and burst events;

FIG. 2H shows an exemplary summary of percent of thalamic spikes in bursts with and without VNS;

FIG. 2I shows an exemplary summary of information transmission efficiency (bits/spike) with standard duty-cycle VNS;

FIG. 2J shows an exemplary summary of information transmission rate (bits/second) with standard duty-cycle VNS;

FIG. 3A shows exemplary feature selectivity of an example VPm neuron recovered during the different periods of standard duty-cycle VNS (inset shows corresponding nonlinear tuning function);

FIG. 3B shows an exemplary summary of feature modulation factor during the different periods of standard duty-cycle VNS;

FIG. 3C shows an exemplary summary of fraction of spikes during the different periods of standard duty-cycle VNS;

FIG. 3D shows an exemplary summary of improvement in information transmission during the different periods of standard duty-cycle VNS;

FIG. 4A shows an exemplary summary of VPm firing rate in response to the same whisker stimulation referenced above during the varying patterns;

FIG. 4B shows an exemplary summary of feature modulation factor during the different VNS patterns;

FIG. 4C shows an exemplary summary of improvement in information transmission efficiency (bits/spike) during the different VNS patterns;

FIG. 4D shows an exemplary summary of fraction of spikes in bursts during the different VNS patterns;

FIG. 4E shows an exemplary summary of information transmission efficiency (bits/spike) with different VNS patterns;

FIG. 4F shows an exemplary summary of firing rate during the different periods of fast duty-cycle VNS;

FIG. 4G shows an exemplary summary of fraction of spikes factor during the different periods of fast duty-cycle VNS;

FIG. 4H shows an exemplary summary of feature modulation during the different periods of fast duty-cycle VNS;

FIG. 4I shows an exemplary summary of improvement of information transmission efficiency during the different periods of fast duty-cycle VNS;

FIG. 5A shows an exemplary summary of VPm firing rate during varying amplitudes of fast duty-cycle VNS;

FIG. 5B shows an exemplary feature selectivity of an example VPm neuron recovered during varying amplitudes of fast duty-cycle VNS;

FIG. 5C shows an exemplary summary of feature modulation factor during varying amplitudes of fast duty-cycle VNS;

FIG. 5D shows an exemplary summary of improvement in information transmission during varying amplitudes of fast duty-cycle VNS;

FIG. 5E shows an exemplary summary of fraction of spikes in bursts during varying amplitudes of fast duty-cycle VNS;

FIG. 5F shows an exemplary summary of VPm firing rate during varying amplitudes of tonic VNS;

FIG. 5G shown an exemplary feature selectivity of an example VPm neuron recovered during varying amplitudes of tonic VNS (inset shows corresponding nonlinear tuning function);

FIG. 5H shows an exemplary summary of feature modulation factor during varying amplitudes of tonic VNS;

FIG. 5I shows an exemplary summary of improvement in information transmission efficiency during varying amplitudes of tonic VNS;

FIG. 5J shows an exemplary summary of fraction of spikes in bursts during varying amplitudes of tonic VNS;

FIG. 6A shows an exemplary summary of VPm firing rate during varying frequencies of tonic VNS;

FIG. 6B shows an exemplary summary of fraction of spikes in bursts during varying frequencies of tonic VNS;

FIG. 6C shows an exemplary summary of feature selectivity of an example VPm neuron recovered during varying frequencies of tonic VNS (inset shows corresponding nonlinear tuning function;

FIG. 6D shows an exemplary summary of feature modulation factor during varying frequencies of tonic VNS;

FIG. 6E shows an exemplary summary of improvement in information transmission efficiency during varying frequencies of tonic VNS;

FIG. 7A shows an exemplary summary of perievent spike raster of the same neurons response to multiple presentations of the same frozen WGN stimulus, with responses during 5 Hz LC activation (yellow dots) overlaid on top of responses during control conditions (blue dots) (top), and corresponding SDFs of the above responses for both control and 5 Hz LC activation, dotted lines indicate event threshold;

FIG. 7B shows an exemplary summary of average events/sec that are classified as removed events during control conditions (2.6 plus or minus 0.2 Hz) (left) and average events/sec that are classified as emerged events during 5 Hz LC activation (1.9 plus or minus 0.2 Hz) (right);

FIG. 7C shows an exemplary summary of percent of all control events that are removed during 5 Hz LC activation (50 plus or minus 4 percent) (left) and percent of all events classified as emerged events during 5 Hz LC activation (40 plus or minus 3 percent) (right);

FIG. 8A shows an example of recovered feature selectivity for VPm spikes falling in different event types;

FIG. 8B shows exemplary non-linear tuning functions corresponding to the feature selectivity in FIG. 8A;

FIG. 8C shows an exemplary population average of feature modulation factor for spikes falling within removed events vs. spikes falling within emerged events;

FIG. 8D shows an exemplary population average of information transmission efficiency for spikes falling within removed events vs. spikes falling within emerged events;

FIG. 8E shows an exemplary population average of feature modulation factor for spikes falling within conserved events without LC stimulation vs. spikes falling within conserved events with 5Hz LC stimulation;

FIG. 8F shows an exemplary population average of information transmission efficiency for spikes falling within conserved events without LC stimulation vs. spikes falling within conserved events with 5 Hz LC stimulation;

FIG. 8G shows an exemplary population average of information transmission efficiency for spikes falling within removed events vs. spikes falling within emerged events;

FIG. 8H shows an exemplary population average of information transmission efficiency for spikes falling within conserved events without LC stimulation vs. spikes falling within conserved events with 5 Hz LC stimulation;

FIG. 8I shows an exemplary population average of information transmission efficiency for spikes falling within removed events vs. spikes falling within emerged events;

FIG. 8J shows an exemplary population average of information transmission efficiency for spikes falling within conserved events without LC stimulation vs. spikes falling within conserved events with 5 Hz LC stimulation;

FIG. 9A shows an example of feature coefficient value over time for a specific neuron and directional feature selectivity (left top) (red stars indicate the peaks with the largest positive value) and SDF of the same neuron's actual response to the whisker stimulus (left bottom)(blue stars indicated observed events) and directionally selective feature corresponding to the panels (right);

FIG. 9B shows an example of the feature coefficient value over time for a specific neuron and non-directional feature selectivity (left top) (red stars indicate the peaks with the largest absolute value) and SDF of the same neuron's actual response to the whisker stimulus (left bottom) (blue stars) and non-directionally selective feature corresponding to the panels (right);

FIG. 9C shows exemplary fraction of events occurring at “ideal” timepoints with and without LC stimulation at 5 Hz;

FIG. 9D shows an exemplary population average of directionality of nonlinear tuning functions corresponding to significant feature selectivity with and without 5 Hz LC stimulation

FIG. 10A shows an example of original versus reconstructed whisker deflection stimulus with and without LC stimulation;

FIG. 10B shows an example the correlation coefficient between and original and reconstructed stimulus versus the number of features used for reconstruction with and without LC stimulation;

FIG. 10C shows an example of RMSE (root mean square error) between original and reconstructed stimulus versus the number of features used for reconstruction;

FIG. 11A shows an example of TRN neuron with significant feature selectivity, within and without LC stimulation;

FIG. 11B shows exemplary nonlinear tuning functions corresponding to the feature selectivity of FIG. 11A;

FIG. 11C shows an example of TRN neuron with significant feature selectivity during LC stimulation that lacked significant feature selectivity without LC stimulation; and

FIG. 11D shows exemplary nonlinear tuning functions corresponding to the feature selectivity of FIG. 11C.

### DETAILED DESCRIPTION

Certain data disclosed herein was published after the earliest priority date of this application. Rodenkirch et. al., Rapid and transient enhancement of thalamic information transmission induced by vagus nerve stimulation, J. Neural Eng. 17 026027, (Apr. 8, 2020).

Aspects described herein provide bioelectronic methods of and devices for improving perceptual acuity based on arousal-linked neuromodulation of sensory processing. Methods of using peripheral stimulation of the vagus nerve to induce neuromodulation that sharpens sensory acuity through optimizing sensory processing are provided. Devices described herein can be externally worn, transcutaneous vagus nerve stimulators (nVNS).

In some aspects, the devices are lightweight, noninvasive neural interface that can be easily taken on and off, allowing users to engage the device during important moments. For example, nVNS can be used during social situations where ability to communicate clearly is key or when working in potentially dangerous conditions or with potentially dangerous equipment. In some instances, the devices are noninvasive, with electrical current being delivered to the vagus nerve through the skin by, for example, an external adhesive flat electrode patch resting above where the vagus nerve runs through neck.

Methods and devices described herein improve current methods of modifying sensory processing, including stimulants and nootropics. nVNS is well-known to be a safe and effective method of inducing neuromodulation unlike addictive stimulants that cause cardiac damage and insomnia and nootropics which lack long-term safety testing. In some instances, the underlying mechanism of action for the methods and devices described herein can provide full strength of effect seconds after activation and the effect remains constant until deactivation. The methods and devices described herein can be used in an on-demand, task dependent manner unlike orally administered stimulants whose effect cannot be rapidly switched on and off.

Aspects described herein provide methods of modifying sensory processing in a subject by applying a tonic vagus nerve stimulation to the subject wherein the sensory processing of the subject is modified. The term “tonic” refers to sustained or graded stimulation or a sufficiently rapid duty cycle stimulation. In some instances, a tonic vagus nerve stimulation does not contain periods of quiescence longer than about 10 seconds.

Previous implanted VNS devices have maximum speed duty cycle that has a period of quiescence (i.e., off cycle) of 12 seconds. In some instances, aspects descried herein have a period of quiescence not greater than about 10 or 11 seconds. Without being bound by theory, it is believed that the effects on sensory processing fade after a long period of quiescence, which creates a fluctuating bias on sensory processing. See, e.g., Paragraphs [00239]-[00241], [00223]-[00250], [00254]-[00257] herein.

Previous gammacore transcutaneous VNS devices use a continuous pattern to treat cluster headaches and migraines. However, the VNS is delivered without any quiescence periods, so the VNS is delivered for only up to 3 minutes before stopping to prevent damage. These previous devices are designed to deliver 3 minutes of stimulation at a time spaced out by hour intervals. In contrast, aspects described herein deliver continuous stimulation a task that may exceed 3 minutes and thus the previous devices would not be suitable for these aspects. Further aspects include periods of quiescence in the VNS stimulation to prevent charge build up. Therefore, in another aspect, fast duty-cycle VNS can be used for enhancing sensory acuity.

The term “modifying sensory processing” refers to changing sensory processing (e.g., vision, hearing, smell, taste, touch etc.) in a subject. In one aspect, the modification is improving sensory processing such that the subject performs tasks in an improved manner (e.g., faster, more accurate, more safely, or for a longer period of time).

In one aspect, the frequency of the vagus nerve stimulation is at least about 0.3 Hz, between about 0.5 and 80 Hz, or between about 30 and 60 Hz. See, e.g., Paragraphs [00242]-[00270] herein. In some instances, the vagus nerve stimulation pulse structure is selected from the group consisting of one or more cycles of single biphasic square pulse, asymmetric biphasic pulse, triangle biphasic pulse, gaussian biphasic pulse, interphase gap biphasic pulse, psuedomonophasic pulse, sinusodial pulse.

In some instances, at least about 0.2 mA, about 0.5 to about 3 mA, or about 1.5 to about 2.5 mA of a current of the vagus nerve stimulation reaches the vagus nerve. See, e.g., Paragraphs [00234]-[00264] herein.

In some instances, a time of applying the tonic vagus nerve stimulation is at least about 3 seconds, at least about 30 seconds, or at least about 4 minutes.

The sensory processing is modified by the methods described herein within less than about 1 second, about less than 10 seconds, or less than about 1 minute. The modified sensory processing can be transient. The term “transient” refers a period of time that is not permanent. In some instances, the period of time can be brief or short (e.g., dissipating within about 5 seconds, 30 seconds, or 1 minute). See, e.g., Paragraphs [00239]-[00241] herein.

The vagus nerve stimulation can be continuous or discontinuous. The term “continuous” refers to without interruption and the term “discontinuous” refers to with interruption.

The discontinuous vagus nerve stimulation can be in the form of a duty cycle. The term “duty cycle” refers to a period of time for a signal to complete an on-off cycle. In some instances, the portion of the duty cycle when vagus nerve stimulation is not applied is not greater than about 7 to about 10 seconds. In one aspect, the portion of the duty cycle when vagus nerve stimulation is not applied is not greater than about 3 to 7 seconds. In another aspect, the portion of the duty cycle when vagus nerve stimulation is not applied is not greater than about 0.5 to 3 seconds. See, e.g., Paragraphs [00239]-[00241], [00223]-[00250], [00254]-[00257] herein.

In some instances, the modifying of sensory processing increases sensory acuity or perceptual sensitivity. The term “sensory acuity” refers to the ability of one or more senses to accurately interpret a signal. In some instances, increasing of the sensory acuity comprises enhancing the acuity of a sensory modality (e.g., visual, auditory, olfactory, gustatory, and tactile stimuli).

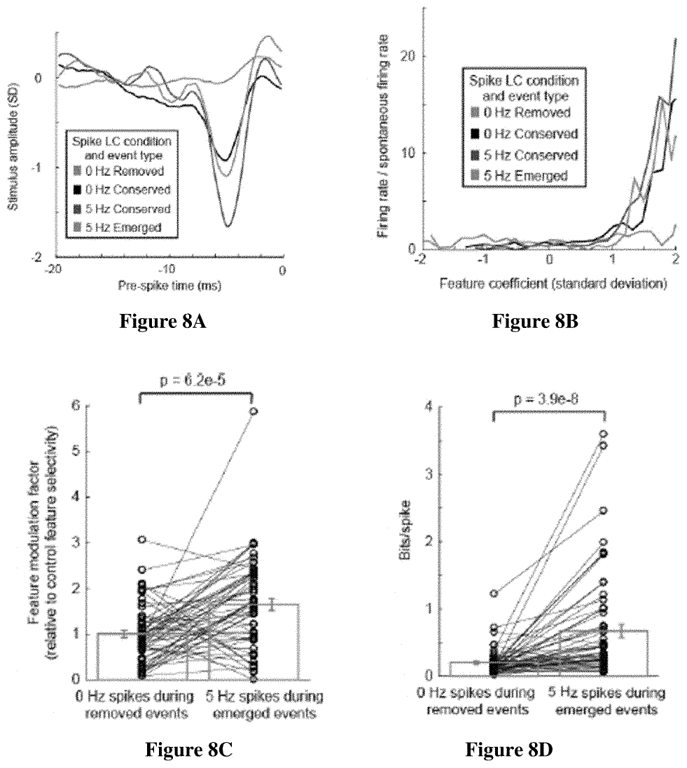

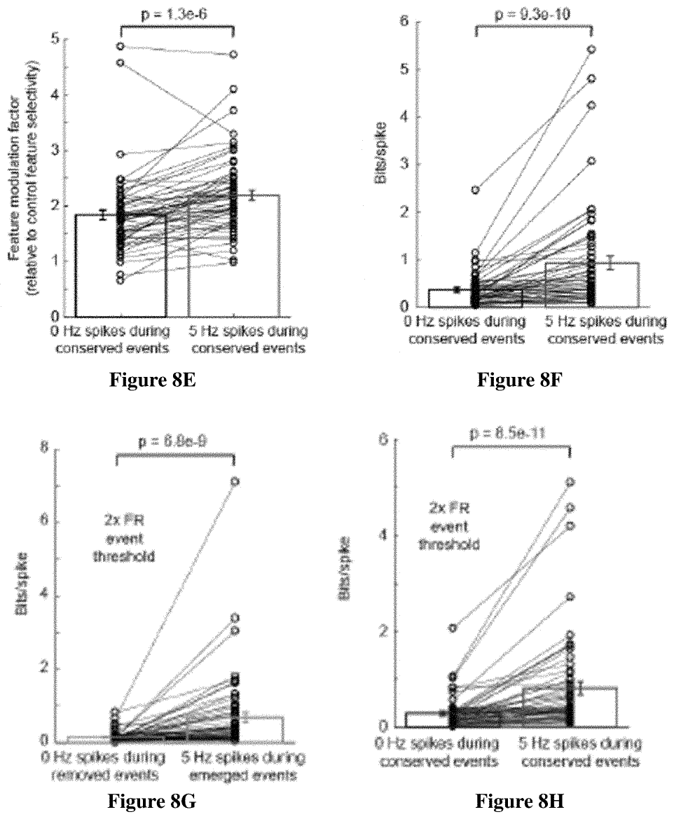

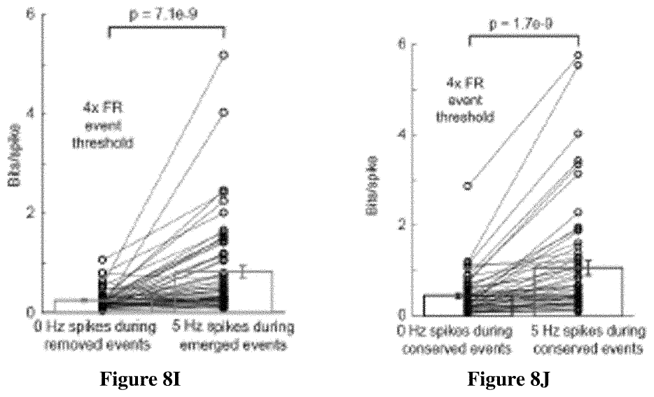

In some instances, the modifying of sensory processing comprises reducing misperception-induced errors. See, e.g., Rodenkirch et al., Locus coeruleus activation enhances thalamic feature selectivity via norepinephrine regulation of intrathalamic circuit dynamics, Nature Neuroscience, vol. 22 (January 2019), FIG. 8, page 130 and accompanying text. In another aspect, the modifying sensory processing comprises selective activation of the Locus Coeruleus.

In some instances, the modifying of sensory processing comprises altering the temporal structure of neural activity used to encode a stimulus. See, e.g., Paragraphs [00275]-[00279], [00256]-[00289], [00266]-[00299] herein.

In some instances, the modifying of sensory processing facilitates the writing of information to the brain by brain-machine interface (e.g. patterned microstimulation used by sensory neuroprosthetics, augmented/virtual reality applied directly to sensory pathways).

In one aspect, the modifying of sensory processing does not arise from lasting neuroplastic changes. See, e.g., Paragraphs [00239]-[00241] herein.

In another aspect, the modifying of sensory processing improves the ability to perform multisensory integration (e.g., using two or more senses in combination such as using both visual and tactile feedback to catch a ball). Improving the ability to perform multi-sensory integration can be measured, for example, by an increase in sensory acuity in two or more senses which can be quantified by an increase in perceptual sensitivity on tasks which may require simultaneous use of two or more senses.

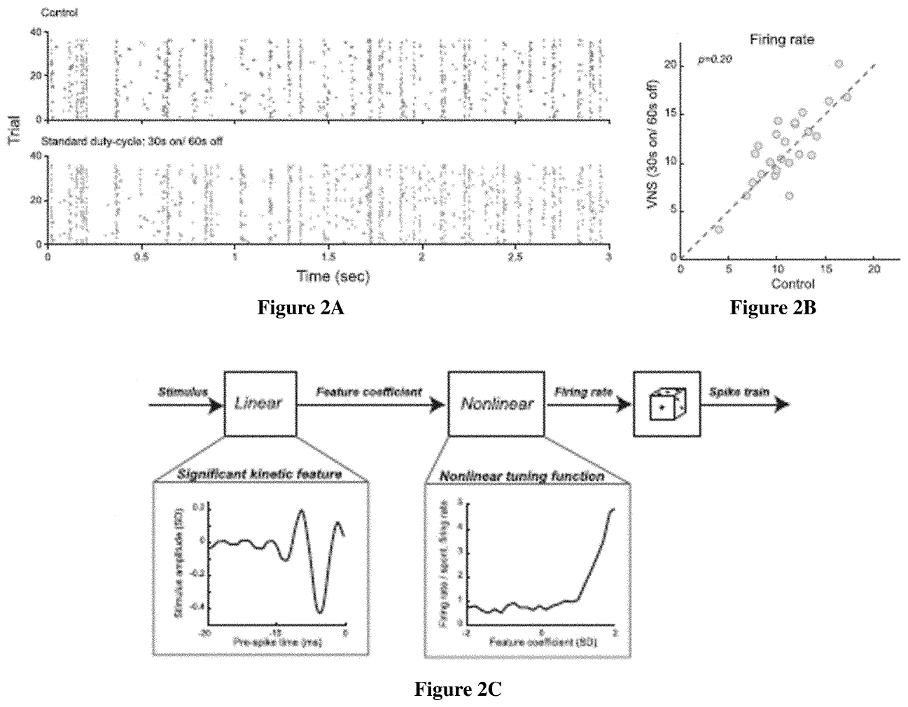

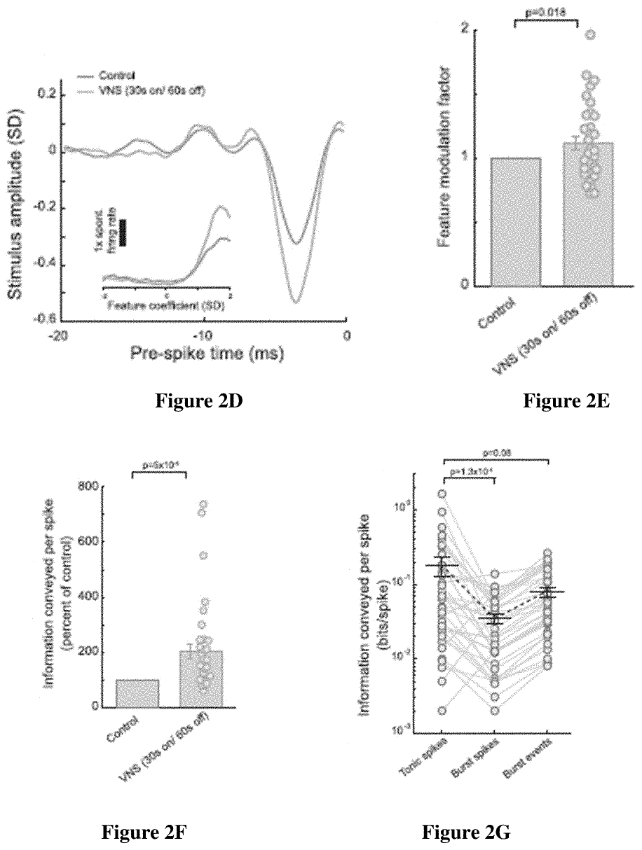

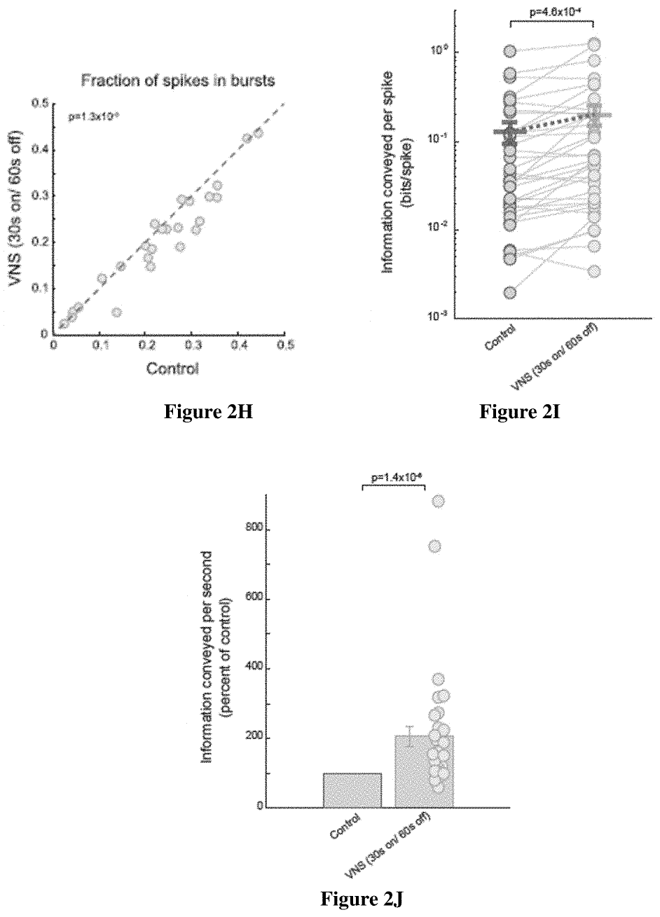

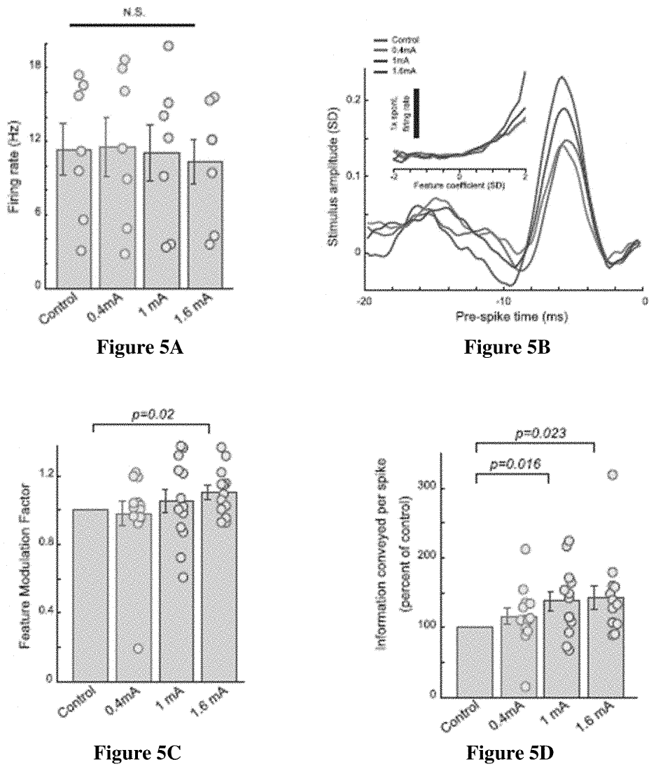

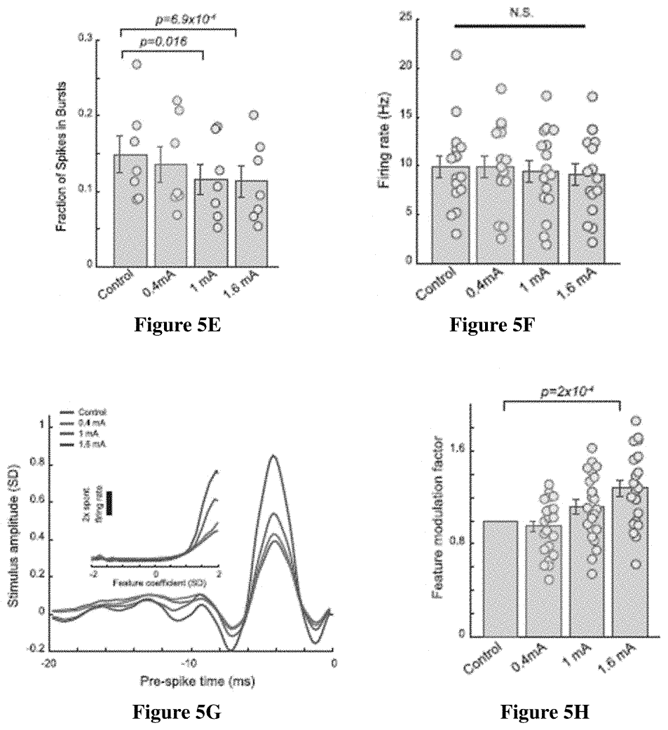

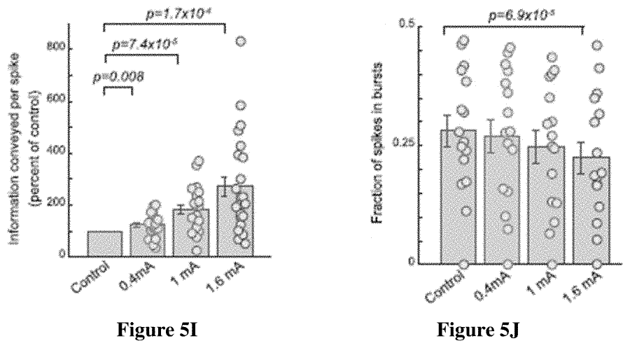

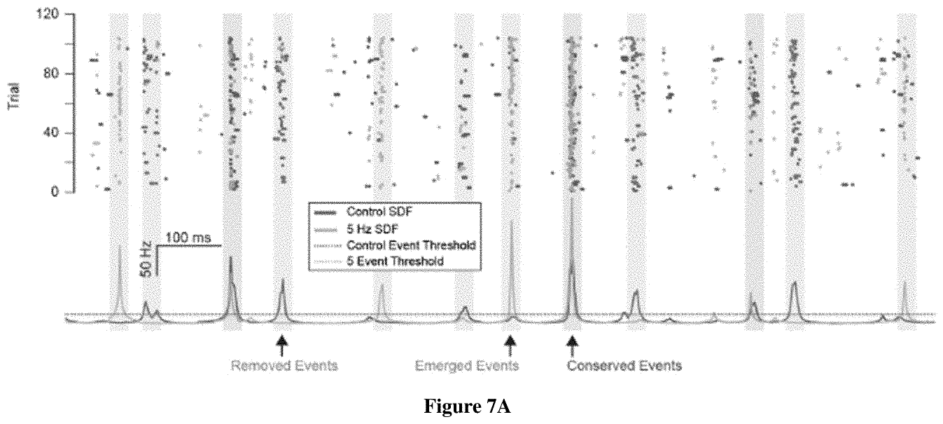

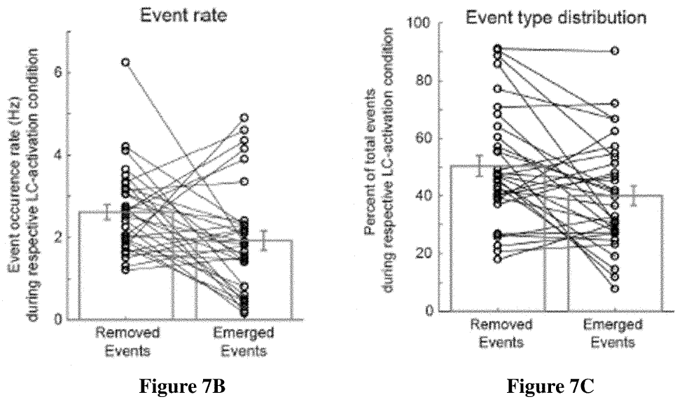

In some instances, the modifying of sensory processing arises due to neuromodulation which reduces calcium t-channel activity. Calcium t-channels are responsible for burst spiking activity. Calcium t-channel influence, and the resulting calcium t-channel induced burst spiking activity, was found to degrade the efficiency and rate of information transmitted by thalamocortical sensory neurons. LC stimulation and VNS decrease bursting activity. LC stimulation decreased bursting rate by ˜60% and it is estimated that calcium t-channel current contributions to thalamic spiking decrease by ˜25% with LC stimulation. See, e.g., Rodenkirch et al., Locus coeruleus activation enhances thalamic feature selectivity via norepinephrine regulation of intrathalamic circuit dynamics, Nature Neuroscience, vol. 22 (January 2019), (FIGS. 5, 6, 7) and accompanying text. VNS decreases the probability of a spike being in a burst by ˜10 to 25%. See, e.g., Rodenkirch et. al., Rapid and transient enhancement of thalamic information transmission induced by vagus nerve stimulation, J. Neural Eng. 17 026027 (FIGS. 2h, 7e,j and 8e) and accompanying text.

In further aspects, the modifying of sensory processing reduces the occurrence of sensory perception that is uncomfortable or distracting (e.g., in individuals with sensory processing disorders that can make certain auditory, visual, gustatory, olfactory, or tactile stimulation uncomfortable, painful, overwhelming, or distracting).

In some instances, the modifying of sensory processing selectively favors a specific sensory modality (e.g., modification is stronger for one sense versus another sense—tactile versus auditory).

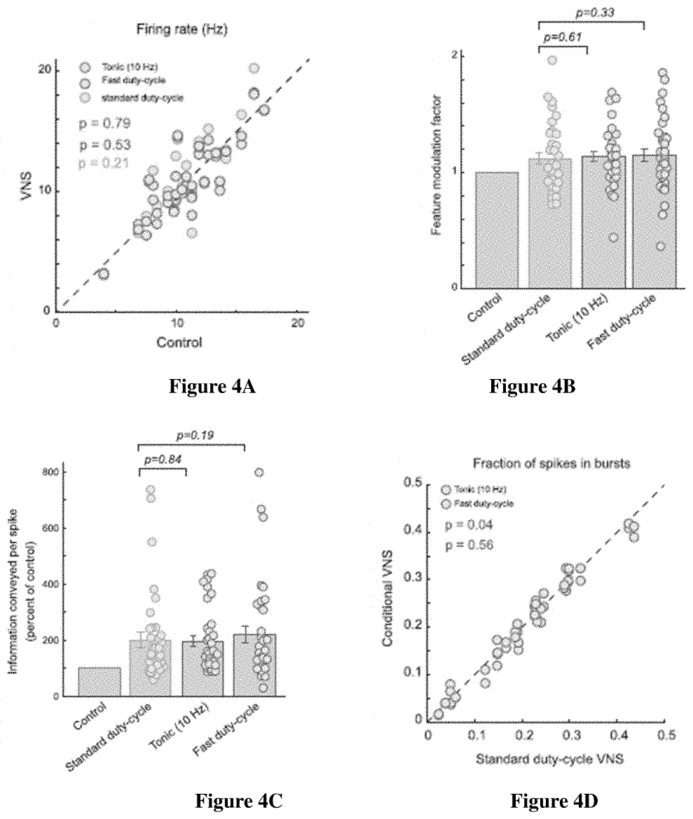

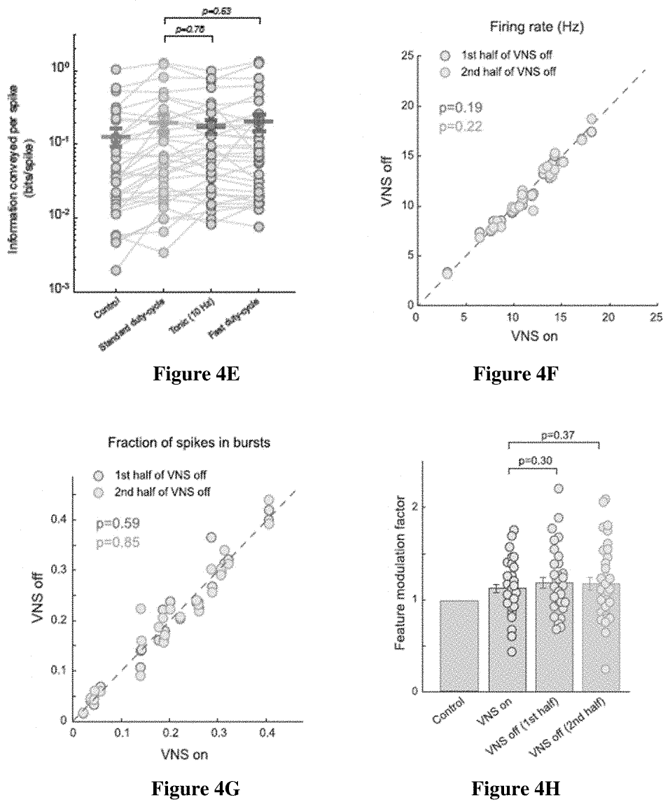

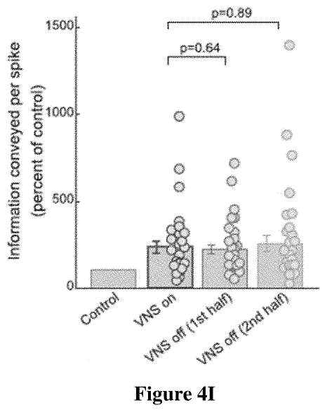

In further aspects, the modifying of sensory processing comprises increasing norepinephrine concentration in the sensory pathway portions of the brain (e.g., thalamus, cortex). See, e.g., Rodenkirch et. al., Locus coeruleus activation enhances thalamic feature selectivity via norepinephrine regulation of intrathalamic circuit dynamics, Nature Neuroscience, vol. 22 (January 2019), (FIG. 4) and accompanying text.

In another aspect, the efficiency of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [0024]-[00270] herein. The term “increased information transmission efficiency” refers to the efficiency of the transfer of information by a sensory neuron in regards to the information (i.e. bits) a each spike of a neuron's spiking response encodes about the absence/presence of a feature in the stimulus similar (i.e. mutual information between stimulus and spike train).

In yet another aspect, a rate of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein.

In a further aspect, the correlation coefficient between an original stimulus and a reconstructed stimulus is increased on average by at least about 10%, or at least about 20%, or by about 25% to 60%, compared to a subject that does not receive vagus nerve stimulation. See, e.g., Paragraphs [00277]-[00311] herein.

In some instances, the vagus nerve stimulation is not paired with a sensory stimulation one or more times. Previously, bursts of VNS has been applied by pairing the VNS with another stimuli (i.e., a tactile stimuli (fingerpad tap) or a audio stimuli (frequency tone)) over a long period of time.^{20, 37-46}. This method can improve detection of the particular paired stimuli after a period of time and is neuroplasticity-based. The previous methods do not improve sensory acuity generally or for stimuli beyond the paired stimulus.

In accordance with aspects described herein, the VNS can be applied to any suitable location in order to modify sensory processing. In some instances, the vagus nerve stimulation is applied to a cervical region of the subject (e.g., left cervical region, right cervical region of the subject or both). In some instances, the vagus nerve stimulation is applied to the auricular transcutaneous region (left auricular transcutaneous region, right auricular transcutaneous region of the subject or both).

In some instances, the modifying of sensory processing comprises improving sensory perception in a subject having one or more impaired senses (e.g., a visual impairment, an auditory impairment, a tactile impairment, an olfaction impairment, and a gustatory impairment).

In some aspects the subject does not have an impairment condition in need of sensory modification (e.g., a visual impairment, an auditory impairment, a tactile impairment, an olfaction impairment, and a gustatory impairment). For example, such a subject might be considered to be generally healthy.

Further aspects provide methods of modifying sensory processing in a subject, by determining a mean value and a variance value for the pupil diameter from the first time point to the second time point; measuring the pupil diameter and determining a pupil diameter value; and applying tonic vagus nerve stimulation to the subject when the pupil diameter value is at least about one to three standard deviations from the mean value for pupil diameter. In some instances, the subject is exposed to a sensory stimulation.

In this aspect, pupil diameter can be measured with modified eyewear or a camera (e.g., webcam, contact lens, and eye implant). For example, a subject (e.g., air traffic control personnel) can wear modified glasses (e.g., Google glass or similar device) that monitors pupil diameter during a series of tasks. Pupil diameter can be calibrated by calculating a mean and variance value for pupil diameter from a first time point to a second time point. Periodic measurements can be taken during a series of tasks. When pupil diameter is at least about one to three standard deviations from the mean value, vagus nerve stimulation can be applied as described herein for a desired period of time (e.g., 1, 4, 5, 10, 15, 30, 45, 60, 90 seconds etc.) or continuously during a given task (e.g., guiding the landing of a plane).

In another aspect, pupil diameter or another proxy for reduced sensory processing can be measured by an algorithm or machine learning method to determine when VNS stimulation is needed and the length of time for VNS treatment. Alternatively, the length of time can be predetermined for a given task.

In one aspect, the frequency of the vagus nerve stimulation is at least about 0.3 Hz, between about 0.5 and 80 Hz, or between about 30 and 60 Hz. See, e.g., Paragraphs [00242]-[00270] herein.

In some instances, at least about 0.2 mA, about 0.5 to about 3 mA, or about 1.5 to about 2.5 mA of a current of the vagus nerve stimulation reaches the vagus nerve. See, e.g., Paragraphs [00234]-[00241] herein. In another aspect, about 1 to about 60 mA or 5 to about 30 mA of a current leaves a device generating the vagus nerve stimulation.

In some instances, a time of applying the tonic vagus nerve stimulation is at least about 3 seconds, at least about 30 seconds, or at least about 4 minutes.

The sensory processing is modified by the methods described herein within less than about 1 second, about less than 10 seconds, or less than about 1 minute. The modified sensory processing can be transient. The term “transient” refers a period of time that is not permanent. In some instances, the period of time can be brief or short (e.g., dissipating within about 5 seconds, 30 seconds, or 1 minute). See, e.g., Paragraphs [00239]-[00241] herein.

The vagus nerve stimulation can be continuous or discontinuous. The term “continuous” refers to without interruption and the term “discontinuous” refers to with interruption.

The discontinuous vagus nerve stimulation can be in the form of a duty cycle. The term “duty cycle” refers to a period of time for a signal to complete and on-off cycle. In some instances, the portion of the duty cycle when vagus nerve stimulation is not applied is not greater than about 7 to about 10 seconds. In one aspect, the portion of the duty cycle when vagus nerve stimulation is not applied is not greater than about 3 to 7 seconds. In another aspect, the portion of the duty cycle when vagus nerve stimulation is not applied is not greater than about 0.5 to 3 seconds. See, e.g., Paragraphs [00239]-[00241], [00223]-[00250], [00254]-[00257] herein.

In some instances, the modifying of sensory processing increases sensory acuity. The term “sensory acuity” refers to the ability of one or more senses to accurately interpret a signal. In some instances, increasing of the sensory acuity comprises enhancing the acuity of a sensory modality (e.g., visual, auditory, olfactory, gustatory, and tactile stimuli). Increased perceptual sensitivity is a widely accepted measure of increased sensory acuity.

In some instances, the modifying of sensory processing comprises reducing misperception-induced errors. See, e.g., Rodenkirch et al., Locus coeruleus activation enhances thalamic feature selectivity via norepinephrine regulation of intrathalamic circuit dynamics, Nature Neuroscience, vol. 22 (January 2019), FIG. 8, page 130 and accompanying text. In another aspect, the modifying sensory processing comprises selective activation of the Locus Coeruleus.

In some instances, the modifying of sensory processing comprises altering the temporal structure of neural activity used to encode a stimulus. See, e.g., Paragraphs [00275]-[00279], [00256]-[00289], [00266]-[00276] herein.

In some instances, the modifying of sensory processing facilitates the writing of information to the brain by brain-machine interface (e.g. patterned microstimulation used by sensory neuroprosthetics, augmented/virtual reality applied directly to sensory pathways).

In one aspect, the modifying of sensory processing does not arise from neuroplastic changes. See, e.g., Paragraphs [00239]-[00241] herein.

In another aspect, the modifying of sensory processing improves the ability to perform multisensory integration (e.g., using two or more senses in combination such as using both visual and tactile feedback to catch a ball). Improving the ability to perform multi-sensory integration can be measured, for example, by an increase in sensory acuity in two or more senses which can be quantified by an increase in perceptual sensitivity on tasks which require simultaneous use of two or more senses.

In some instances, the modifying of sensory processing arises due to neuromodulation which reduces calcium t-channel activity. Calcium t-channels are responsible for burst spiking activity. Calcium t-channel influence, and the resulting calcium t-channel induced burst spiking activity, was found to degrade the efficiency and rate of information transmitted by thalamocortical sensory neurons. LC stimulation and VNS decrease bursting activity. LC stimulation decreased bursting rate by -60% and it is estimated that calcium t-channel current contributions to thalamic spiking decrease by ˜25% with LC stimulation. See, e.g., Rodenkirch et al., Locus coeruleus activation enhances thalamic feature selectivity via norepinephrine regulation of intrathalamic circuit dynamics, Nature Neuroscience, vol. 22 (January 2019), (FIGS. 5, 6, 7) and accompanying text. VNS decreases the probability of a spike being in a burst by ˜10 to 25%. See, e.g., Rodenkirch et. al., Rapid and transient enhancement of thalamic information transmission induced by vagus nerve stimulation, J. Neural Eng. 17 026027 (FIGS. 2h, 7e,j and 8e) and accompanying text.

In further aspects, the modifying of sensory processing reduces the occurrence of sensory perception that is uncomfortable or distracting (e.g., in individuals with sensory processing disorder that can make certain auditory, visual, gustatory, olfactory or tactile stimulation uncomfortable, painful, overwhelming, or distracting).

In some instances, the modifying of sensory processing selectively favors a specific sensory modality (e.g., modification is stronger for one sense versus another sense≥tactile versus auditory).

In further aspects, the modifying of sensory processing comprises increasing norepinephrine concentration in the sensory pathway portions of the brain (e.g., thalamus, cortex). See, e.g., Rodenkirch et al., Locus coeruleus activation enhances thalamic feature selectivity via norepinephrine regulation of intrathalamic circuit dynamics, Nature Neuroscience, vol. 22 (January 2019), (FIG. 4) and accompanying text.

In another aspect, the efficiency of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein. The term “increased information transmission efficiency” refers to the efficiency of the transfer of information by a sensory neuron in regards to the information (i.e. bits) a each spike of a neuron's spiking response encodes about the absence/presence of a feature in the stimulus similar (i.e. mutual information between stimulus and spike train).

In yet another aspect, a rate of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein.

In a further aspect, the correlation coefficient between an original stimulus and a reconstructed stimulus is increased on average by at least about 10%, or at least about 20%, or by about 25% to 60%, compared to a subject that does not receive vagus nerve stimulation. See, e.g., Paragraphs [00277]-[00311] herein.

In some instances, the vagus nerve stimulation is not paired with a sensory stimulation one or more times.

In accordance with aspects described herein, the VNS can be applied to any suitable location in order to modify sensory processing. In some instances, the vagus nerve stimulation is applied to a cervical region of the subject (e.g., left cervical region, right cervical region of the subject or both). In some instances, the vagus nerve stimulation is applied to the auricular transcutaneous region (left auricular transcutaneous region, right auricular transcutaneous region of the subject or both).

In some instances, the modifying of sensory processing comprises improving sensory perception in a subject having one or more impaired senses (e.g., a visual impairment, an auditory impairment, a tactile impairment, an olfaction impairment, and a gustatory impairment).

In some aspects, the subject does not have an impairment condition in need of sensory modification (e.g., a visual impairment, an auditory impairment, a tactile impairment, an olfaction impairment, and a gustatory impairment). For example, such a subject might be considered to be generally healthy.

Aspects described herein provide methods of modifying sensory processing in a subject by detecting when the subject is in need of a sensory processing modification; applying tonic, vagus nerve stimulation to the subject to provide the sensory processing modification; and discontinuing applying the sensory processing modification when the subject no longer is in need of sensory processing modification.

In one aspect, vagus nerve stimulation (e.g., continuous, tonic vagus nerve stimulation) can be applied when needed and discontinued when the stimulation is not needed. For example, a subject operating performing quality control inspection of a product can have vagus nerve stimulation applied to improve sensory processing only when the product being inspected is present. The presence or absence of an object can be determined, for example, using a camera or other sensory, smart eyewear etc. In another example, a subject performing a task requiring a higher level of concentration (e.g., surgery, flying an airplane, operating heavy machinery) can have vagus nerve stimulation applied to improve sensory processing only when engaged in the task.

In some instances, detecting that the subject is in need of the sensory processing modification comprises determining a mean value and a variance value for the pupil diameter from the first time point to the second time point; measuring the pupil diameter and determining a pupil diameter value; and applying tonic vagus nerve stimulation to the subject when the pupil diameter value is at least about one to three standard deviations from the variance value for pupil diameter.

In one aspect, the frequency of the vagus nerve stimulation is at least about 0.3 Hz, between about 0.5 and 80 Hz, or between about 30 and 60 Hz. See, e.g., Paragraphs [00242]-[00270] herein.

In some instances, at least about 0.2 mA, about 0.5 to about 3 mA, or about 1.5 to about 2.5 mA of a current of the vagus nerve stimulation reaches the vagus nerve. See, e.g., Paragraphs [00234]-[00241] herein. In another aspect, about 1 to about 60 mA or 5 to about 30 mA of a current leaves a device generating the vagus nerve stimulation.

In some instances, a time of applying the tonic vagus nerve stimulation is at least about 3 seconds, at least about 30 seconds, or at least about 4 minutes.

The sensory processing is modified by the methods described herein within less than about 1 second, about less than 10 seconds, or less than about 1 minute. The modified sensory processing can be transient. The term “transient” refers a period of time that is not permanent. In some instances, the period of time can be brief or short (e.g., dissipating within about 5 seconds, 30 seconds, or 1 minute). See, e.g., Paragraphs [00239]-[00241] herein.

The vagus nerve stimulation can be continuous or discontinuous. The term “continuous” refers to without interruption and the term “discontinuous” refers to with interruption.

The discontinuous vagus nerve stimulation can be in the form of a duty cycle. The term “duty cycle” refers to a period of time for a signal to complete and on-off cycle. In some instances, the portion of the duty cycle when vagus nerve stimulation is not applied is not greater than about 7 to about 10 seconds. In one aspect, the portion of the duty cycle when vagus nerve stimulation is not applied is not greater than about 3 to 7 seconds. In another aspect, the portion of the duty cycle when vagus nerve stimulation is not applied is not greater than about 0.5 to 3 seconds. See, e.g., Paragraphs [00239]-[00241], [00223]-[00250], [00254]-[00257] herein.

In some instances, the modifying of sensory processing increases sensory acuity. The term “sensory acuity” refers to the ability of one or more senses to accurately interpret a signal. In some instances, increasing of the sensory acuity comprises enhancing the acuity of a sensory modality (e.g., visual, auditory, olfactory, gustatory, and tactile stimuli). Increased perceptual sensitivity is a widely accepted measure of increased sensory acuity.

In some instances, the modifying of sensory processing comprises reducing misperception-induced errors. See, e.g., Rodenkirch et al., Locus coeruleus activation enhances thalamic feature selectivity via norepinephrine regulation of intrathalamic circuit dynamics, Nature Neuroscience, vol. 22 (January 2019), FIG. 8, page 130. In another aspect, the modifying sensory processing comprises selective activation of the Locus Coeruleus.

In some instances, the modifying of sensory processing comprises altering the temporal structure of neural activity used to encode a stimulus. See, e.g., Paragraphs [00275]-[00279], [00256]-[00289], [00266]-[00276] herein.

In some instances, the modifying of sensory processing facilitates the writing of information to the brain by brain-machine interface (e.g. patterned microstimulation used by sensory neuroprosthetics, augmented/virtual reality applied directly to sensory pathways).

In one aspect, the modifying of sensory processing does not arise from lasting neuroplastic changes. See, e.g., Paragraphs [00239]-[00241] herein.

In another aspect, the modifying of sensory processing improves the ability to perform multisensory integration (e.g., using two or more senses in combination such as using both visual and tactile feedback to catch a ball). Improving the ability to perform multi-sensory integration can be measured, for example, by an increase in sensory acuity in two or more senses which can be quantified by an increase in perceptual sensitivity on tasks which may require simultaneous use of two or more senses.

In some instances, the modifying of sensory processing arises due to neuromodulation which reduces calcium t-channel activity. Calcium t-channels are responsible for burst spiking activity. Calcium t-channel influence, and the resulting calcium t-channel induced burst spiking activity, was found to degrade the efficiency and rate of information transmitted by thalamocortical sensory neurons. LC stimulation and VNS decrease bursting activity. LC stimulation decreased bursting rate by -60% and it is estimated that calcium t-channel current contributions to thalamic spiking decrease by -25% with LC stimulation. See, e.g., Rodenkirch et al., Locus coeruleus activation enhances thalamic feature selectivity via norepinephrine regulation of intrathalamic circuit dynamics, Nature Neuroscience, vol. 22 (January 2019), (FIGS. 5, 6, 7) and accompanying text. VNS decreases the probability of a spike being in a burst by -10 to 25%. See, e.g., Rodenkirch et. al., Rapid and transient enhancement of thalamic information transmission induced by vagus nerve stimulation, J. Neural Eng. 17 026027 (FIGS. 2h, 7e,j and 8e) and accompanying text.

In further aspects, the modifying of sensory processing reduces the occurrence of sensory perception that is uncomfortable or distracting (e.g., in individuals with sensory processing disorder that can make certain auditory, visual, gustatory, olfactory or tactile stimulation uncomfortable, painful, overwhelming, or distracting).

In some instances, the modifying of sensory processing selectively favors a specific sensory modality (e.g., modification is stronger for one sense versus another sense—tactile versus auditory).

In further aspects, the modifying of sensory processing comprises increasing norepinephrine concentration in the sensory pathway portions of the brain (e.g., thalamus, cortex). Rodenkirch et al., Locus coeruleus activation enhances thalamic feature selectivity via norepinephrine regulation of intrathalamic circuit dynamics, Nature Neuroscience, vol. 22 (January 2019), (FIG. 4) and accompanying text.

In another aspect, the efficiency of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein. The term “increased information transmission efficiency” refers to the efficiency of the transfer of information by a sensory neuron in regards to the information (i.e. bits) a each spike of a neuron's spiking response encodes about the absence/presence of a feature in the stimulus similar (i.e. mutual information between stimulus and spike train).

In yet another aspect, a rate of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein.

In a further aspect, the correlation coefficient between an original stimulus and a reconstructed stimulus is increased on average by at least about 10%, or at least about 20%, or by about 25% to 60%, compared to a subject that does not receive vagus nerve stimulation. See, e.g., Paragraphs [00277]-[00311] herein.

In some instances, the vagus nerve stimulation is not paired with a sensory stimulation one or more times. Previously, bursts of VNS have been applied by pairing the VNS with another stimuli (i.e., a tactile stimulus (finger pad tap) or a audio stimuli (frequency tone)) over a long period of time.^{20, 37-46} This method can improve detection of the particular paired stimuli after a period of time and is neuroplasticity-based. The previous methods do not improve sensory acuity generally or for any stimuli.

In accordance with aspects described herein, the VNS can be applied to any suitable location in order to modify sensory processing. In some instances, the vagus nerve stimulation is applied to a cervical region of the subject (e.g., left cervical region, right cervical region of the subject or both). In some instances, the vagus nerve stimulation is applied to the auricular transcutaneous region (left auricular transcutaneous region, right auricular transcutaneous region of the subject or both).

In some instances, the modifying of sensory processing comprises improving sensory perception in a subject having one or more impaired senses (e.g., a visual impairment, an auditory impairment, a tactile impairment, an olfaction impairment, and a gustatory impairment).

In some aspects the subject does not have an impairment condition in need of sensory modification (e.g., a visual impairment, an auditory impairment, a tactile impairment, an olfaction impairment, and a gustatory impairment). For example, such a subject might be considered to be generally healthy.

Further aspects provide a method of modifying sensory processing in a subject, by measuring a change (e.g., sampling a measurement over the time range from a first time point to a second time point) in a bioelectronic signal (e.g., EEG (synchronization, relative power band strength), EKG (heart rate, heart rate variability), change in blood pressure, ECOG, respiratory rate, perspiration (e.g., measured by conductivity of skin surface), or a signal recorded from invasive or noninvasive brain-machine interface) from a first time to a second time; determining a mean value and a variance value for the signal from the first time to the second time; measuring the bioelectronic signal and determining a measured value for the bioelectronic signal; and applying tonic vagus nerve stimulation to the subject when the measured value is at least about one to three standard deviations from the mean value.

Further aspects provide vagus nerve stimulation devices adapted to apply tonic vagus nerve stimulation to a subject to modify sensory processing in the subject, wherein a time of applying the tonic vagus nerve stimulation for at least about 3 seconds, at least about 30 seconds, or at least about 4 minutes. In some aspects, the vagus nerve stimulation is continuous or discontinuous.

The term “adapted to” refers to a device that is configured or programmed to apply vagus nerve stimulation as described herein. For example, the device can include a microprocessor programmed to apply tonic vagus nerve stimulation for at least about 3 seconds, at least about 30 seconds, or at least about 4 minutes and wherein the sensory processing is modified within less than about 1 second, about less than 10 seconds, or less than about 1 minute. The device can be configured or programmed to apply the vagus nerve stimulation in accordance with the methods described herein.

In some instances, the device can be operated manually by a subject in order to apply vagus nerve stimulation on demand. In another aspect, the device can further include a prosthetic device adapted to attach to a body part (i.e., arm, leg, head, torso etc.) and apply vagus nerve stimulation to improve sensory processing to accomplish a particular task.

In some instances, the modifying of sensory processing increases sensory acuity. The term “sensory acuity” refers to the ability of one or more senses to accurately interpret a signal. In some instances, increasing of the sensory acuity comprises enhancing the acuity of a sensory modality (e.g., visual, auditory, olfactory, gustatory, and tactile stimuli). Increased perceptual sensitivity is a widely accepted measure of increased sensory acuity.

In some instances, the modifying of sensory processing comprises reducing misperception-induced errors. See, e.g., Rodenkirch et al., Locus coeruleus activation enhances thalamic feature selectivity via norepinephrine regulation of intrathalamic circuit dynamics, Nature Neuroscience, vol. 22 (January 2019), FIG. 8, page 130. In another aspect, the modifying sensory processing comprises selective activation of the Locus Coeruleus.

In some instances, the modifying of sensory processing comprises altering the temporal structure of neural activity used to encode a stimulus. See, e.g., Paragraphs [00275]-[00279], [00256]-[00289], [00266]-[00276] herein.

In some instances, the modifying of sensory processing facilitates the writing of information to the brain by brain-machine interface (e.g. patterned microstimulation used by sensory neuroprosthetics, augmented/virtual reality applied directly to sensory pathways).

In one aspect, the modifying of sensory processing does not arise from lasting neuroplastic changes. See, e.g., Paragraphs [00239]-[00241] herein.

In another aspect, the modifying of sensory processing improves the ability to perform multisensory integration (e.g., using two or more senses in combination such as using both visual and tactile feedback to catch a ball). Improving the ability to perform multi-sensory integration can be measured, for example, by an increase in sensory acuity in two or more senses which can be quantified by an increase in perceptual sensitivity on tasks which require simultaneous use of two or more senses.

In some instances, the modifying of sensory processing arises due to neuromodulation which reduces calcium t-channel activity. Calcium t-channels are responsible for burst spiking activity. Calcium t-channel influence, and the resulting calcium t-channel induced burst spiking activity, was found to degrade the efficiency and rate of information transmitted by thalamocortical sensory neurons. LC stimulation and VNS decrease bursting activity. LC stimulation decreased bursting rate by ˜60% and it is estimated that calcium t-channel current contributions to thalamic spiking decrease by ˜25% with LC stimulation. See, e.g., Rodenkirch et al., Locus coeruleus activation enhances thalamic feature selectivity via norepinephrine regulation of intrathalamic circuit dynamics, Nature Neuroscience, vol. 22 (January 2019), (FIGS. 5, 6, 7) and accompanying text. VNS decreases the probability of a spike being in a burst by ˜10 to 25%. See, e.g., Rodenkirch et. al., Rapid and transient enhancement of thalamic information transmission induced by vagus nerve stimulation, J. Neural Eng. 17 026027 (FIGS. 2h, 7e,j and 8e) and accompanying text.

In further aspects, the modifying of sensory processing reduces the occurrence of sensory perception that is uncomfortable or distracting (e.g., in individuals with sensory processing disorder that can make certain auditory, visual, gustatory, olfactory, or tactile stimulation uncomfortable, painful, overwhelming, or distracting).

In some instances, the modifying of sensory processing selectively favors a specific sensory modality. (e.g., modification is stronger for one sense versus another sense—tactile versus auditory).

In further aspects, the modifying of sensory processing comprises increasing norepinephrine concentration in the sensory pathway portions of the brain (e.g., thalamus, cortex). See, e.g., Rodenkirch et al., Locus coeruleus activation enhances thalamic feature selectivity via norepinephrine regulation of intrathalamic circuit dynamics, Nature Neuroscience, vol. 22 (January 2019), (FIG. 4) and accompanying text.

The term “increased information transmission efficiency” refers to the efficiency of the transfer of information by a sensory neuron in regards to the information (i.e. bits) a each spike of a neuron's spiking response encodes about the absence/presence of a feature in the stimulus similar (i.e. mutual information between stimulus and spike train).

In yet another aspect, a rate of sensory related information transmitted by a thalamocortical relay neuron in a subject is increased on average by at least about 100 to 200% compared to a subject that does not receive the vagus nerve stimulation. See, e.g., Paragraphs [00218]-[00222], [00227]-[00252], [00234]-[00241], [00242]-[00270] herein.

In a further aspect, the correlation coefficient between an original stimulus and a reconstructed stimulus is increased on average by at least about 10%, or at least about 20%, or by about 25% to 60%, compared to a subject that does not receive vagus nerve stimulation. See, e.g., Paragraphs [00277]-[00311] herein.

In some instances, the vagus nerve stimulation is not paired with a sensory stimulation one or more times.

In accordance with aspects described herein, the VNS can be applied to any suitable location in order to modify sensory processing. In some instances, the vagus nerve stimulation is applied to a cervical region of the subject (e.g., left cervical region, right cervical region of the subject or both). In some instances, the vagus nerve stimulation is applied to the auricular transcutaneous region (left auricular transcutaneous region, right auricular transcutaneous region of the subject or both).

In some instances, the modifying of sensory processing comprises improving sensory perception in a subject having one or more impaired senses (e.g., a visual impairment, an auditory impairment, a tactile impairment, an olfaction impairment, and a gustatory impairment).

In some aspects the subject does not have an impairment condition in need of sensory modification (e.g., a visual impairment, an auditory impairment, a tactile impairment, an olfaction impairment, and a gustatory impairment). For example, such a subject might be considered to be generally healthy.

In some instances, the device is invasive, non-invasive, or minimally invasive. The term “non-invasive” refers to devices and methods of peripheral nerve stimulation that do not require physically penetrating the skin (e.g. transcutaneous, focused ultrasound, vibrational). The term “invasive” refers to devices and methods of peripheral nerve stimulation that may require physically penetrating the skin. “Minimally invasive” methods refer to those that may partially physically penetrate the skin, but in a manner that is painless and safe (e.g. microneedle array surface patch where microneedles slightly penetrate skin without pain or requiring any surgery, and can be easily taken on/off).

Devices described herein can further comprise a prosthetic device adapted to be associated with a body part of the subject in need of vagus nerve stimulation.

In some aspects, the prosthetic device can be adapted to direct the vagus nerve stimulation to a cervical region of the subject. In one aspect, a cervical region comprises a left cervical region, a right cervical region of the subject or both.

In some aspects, the prosthetic device is adapted to direct the vagus nerve stimulation to an auricular transcutaneous region of the subject. In one aspect, a cervical region comprises a left auricular transcutaneous region, a right auricular transcutaneous region of the subject or both.

In some instances, the prosthetic device can be a suitable medical device, article of clothing, or an accessory that can be invasive, non-invasive, or minimally invasive. The prosthetic device can house, be in contact with, or otherwise associated with a vagus nerve stimulating device as described herein. In some instances, the prosthetic device is selected from the group consisting of eyeglasses, sunglasses, a hearing aid, a neck brace, a craniofacial prosthetic, a voice prosthetic (e.g. laryngeal devices), compression stimulation devices (e.g. weighted blankets, or compression style shirts designed to induce neuromodulation), sensory neuroprostheses (e.g. cochlear implant, retina implant, visual cortex implant, auditory cortex implant), an orbital prostheses, a cervical collar, a halo vest, a dental implant, a facial implant, a helmet, a vehicle or machinery cockpit, machinery controls (e.g., a wire running to stimulating patch worn while using the machinery), a head-up display, a headset, a necklace, earrings, goggles (e.g., for athletics or protection), a tiara, a scarf, jewelry, a headdress, a headscarf, a hat, a tie, a bonnet, ear muffs (e.g., for warmth or to protect hearing), headphones, headsets, a shawl, a lanyard, a wig, a hood (e.g., for a shirt or coat), a headband, a hair tie, a barrette, a hair clip, a neck pillow, a shirt collar, a rifle scope, binoculars, a night vision device, a telescope, hair piece, virtual reality headset, phone headset, phone, a video game controller, a video game system, clothing, an adhesive patch, a blood pressure monitor, a heart rate monitor, an oximeter, a watch, a smart watch, a phone, and 3D glasses.

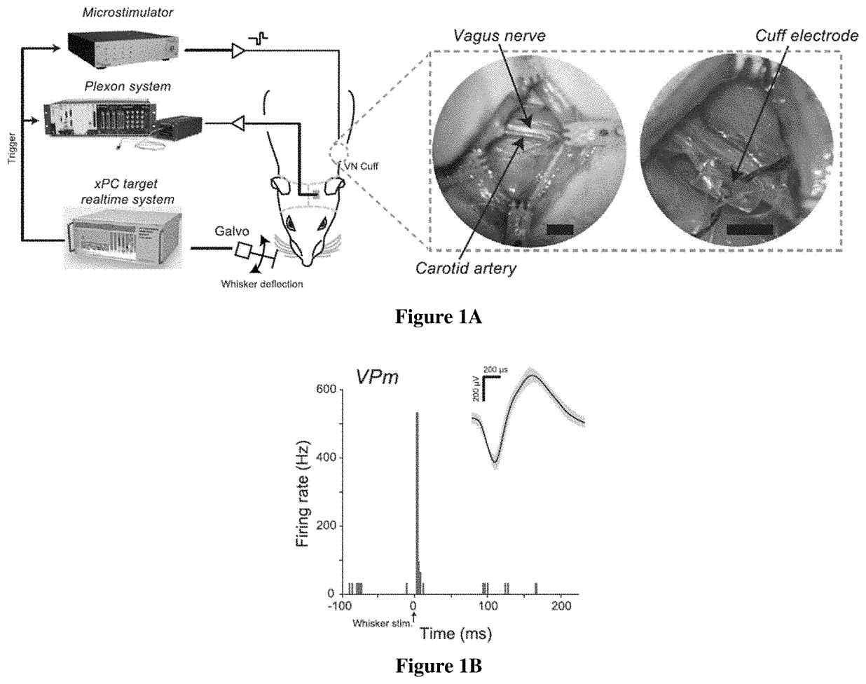

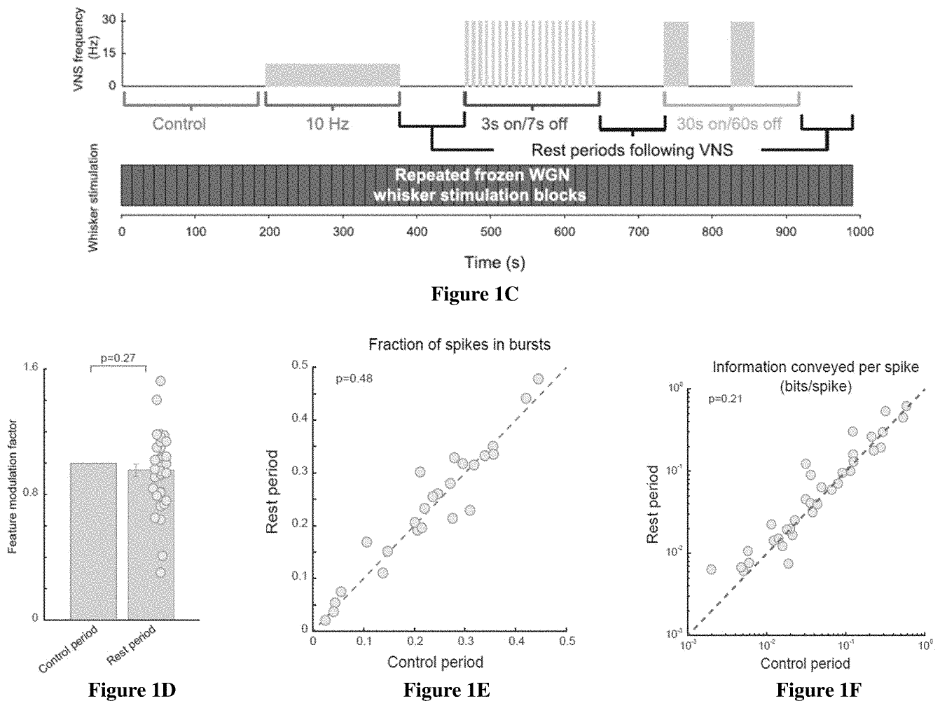

FIGS. 1A-1F provide the results of an exemplary experiment confirming the transient nature of VNS effects on sensory processing using VNS by measuring VNS amplitude, frequency, and sensory neurons response to whisker stimulation during the rest period following VNS (e.g. 45-75 seconds after the cessation of VNS).

FIGS. 2A-2J illustrate that VNS increases feature selectivity and information transmission while also suppressing burst firing.

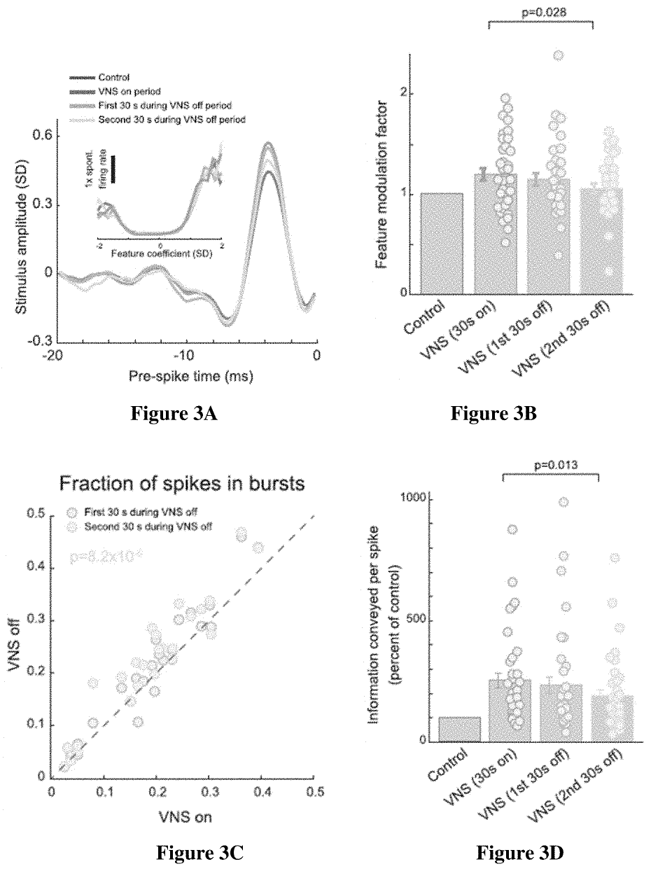

FIGS. 3A-3D illustrate that standard duty cycle VNS (i.e. 30 seconds on/60 seconds off) is suboptimal for optimizing perception as it was observed to create a fluctuating bias in sensory processing state. During the off period the effects of VNS on sensory processing dissipate then return during the next on cycle. This would interfere with discriminating between two stimuli delivered at different periods of the duty-cycled VNS.

FIGS. 4A-4I illustrate that exemplary patterns of tonic and fast duty-cycled VNS (e.g., VNS without a quiescence period greater than about 10 seconds, for example, 3 seconds on/7 seconds off) could be used to enhance sensory processing without creating a fluctuating sensory processing bias. VNS with a fast duty cycle (i.e. 3 seconds on/7 seconds off) enhanced sensory processing without inducing a fluctuating bias while at the same time still containing relatively short periods of quiescence to minimize likelihood of damage to the nerve.

Based on FIGS. 5A-5J, aspects described herein show that increasing the amplitude of tonic VNS and fast duty-cycle VNS (3 sec on/7 sec off) results in increased improvements in sensory processing as evidenced by increased feature selectivity and information transmission. In some instances, these patterns can be optimized to induce a stronger improvement than VNS patterns with long periods of quiescence (i.e. greater than about 10 seconds) that induce a fluctuating bias on sensory processing.

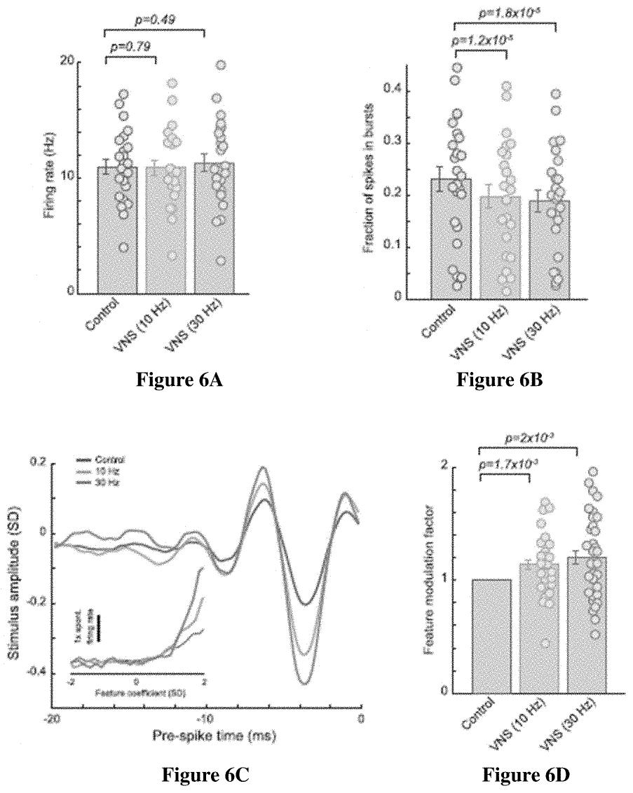

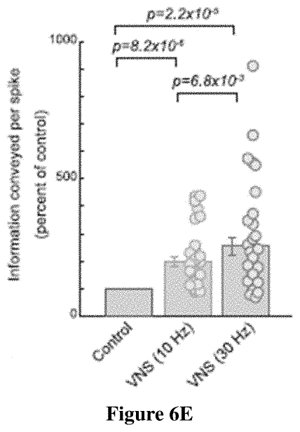

FIGS. 6A-6E illustrate that increasing the frequency of tonic VNS results in increased improvements in sensory processing as evidenced by increased feature selectivity and information transmission. In some instances, continuous tonic 30 Hz VNS improves sensory information transmission rate at about twice the strength of standard duty-cycled VNS, as it does not induce a fluctuating bias on sensory processing.

FIGS. 7A-7C show that exemplary LC-activation can alter the temporal spiking structure thalamocortical sensory relay neurons used to encode the same sensory stimulus.

FIGS. 8A-8J show that exemplary LC-activation-induced alteration of the temporal structure thalamocortical sensory relay neurons used to encode sensory stimulus can generate an encoding system that is more optimal for encoding detailed sensory information (e.g., transmits more sensory-related information per spike and per second, which are efficiency and rate respectively).

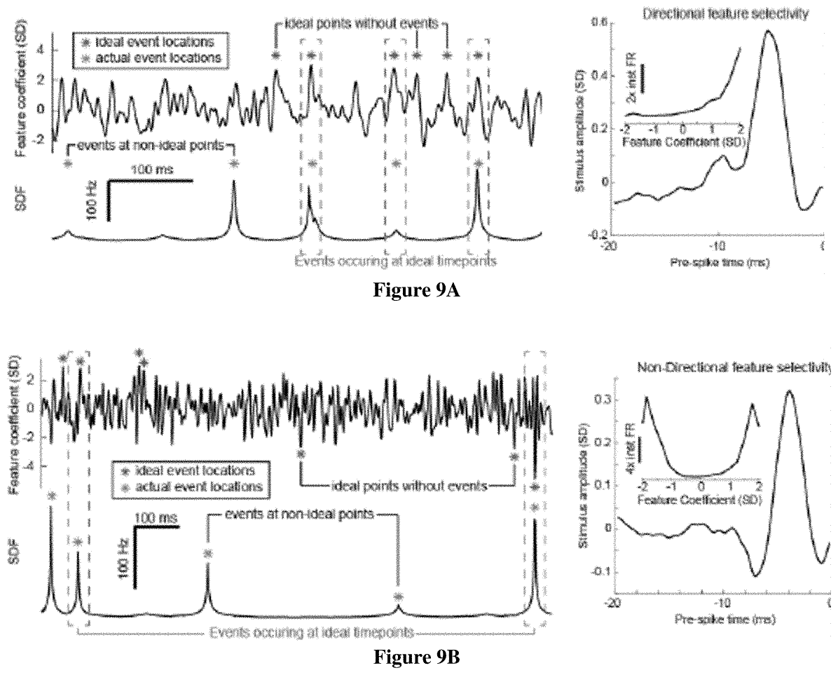

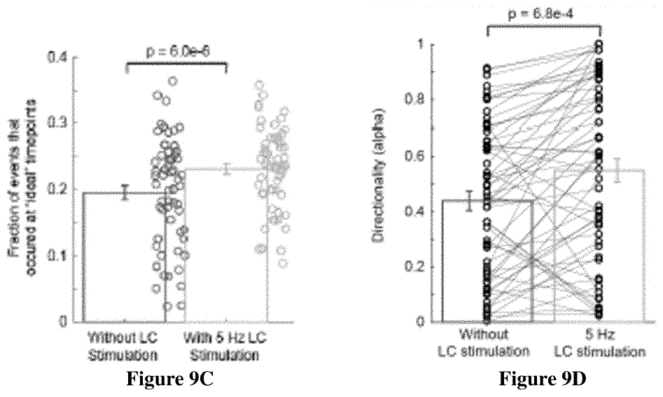

FIGS. 9A-9D show that an example of LC-activation-induced alteration of the temporal structure thalamocortical sensory relay neurons used to encode sensory stimulus is optimal for encoding sensory stimuli. In this example, during LC activation, the neurons more selectively respond to only features in sensory stimuli that most closely match the feature whose presence/absence is encoded. Here, the “feature coefficient” refers to how similar the stimulus is at that timepoint to the neuron-encoded feature. LC activation, in this example, also increases the directional selectivity of thalamocortical sensory relay neurons, indicating that LC activation likely improves the ability to discriminate stimuli direction.

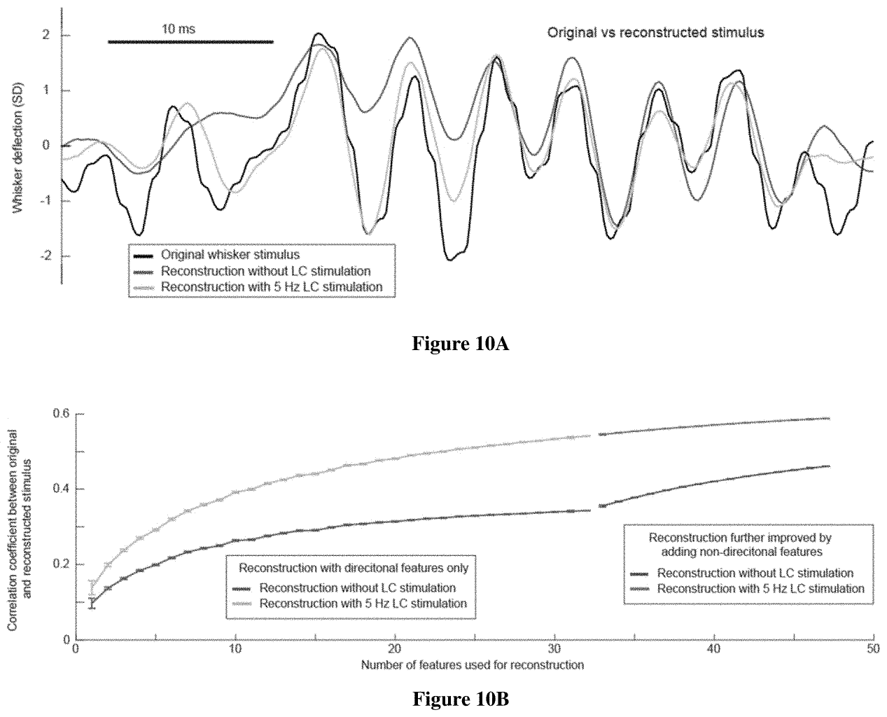

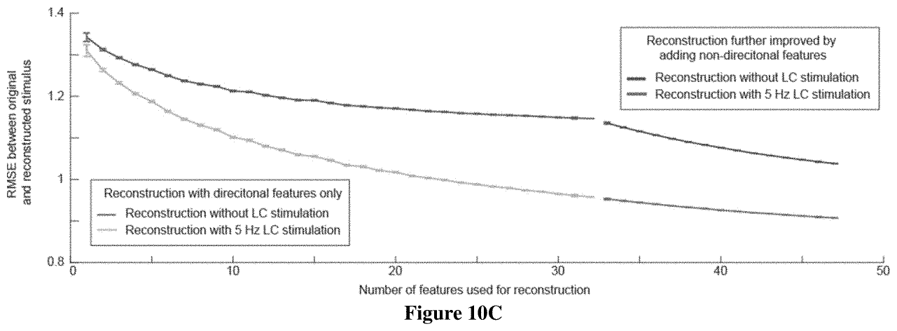

FIGS. 10A-10C show LC-activation-induced improved thalamic information encoding allows for a more accurate reconstruction of the original stimulus from thalamic neurons feature selectivity and spike trains.

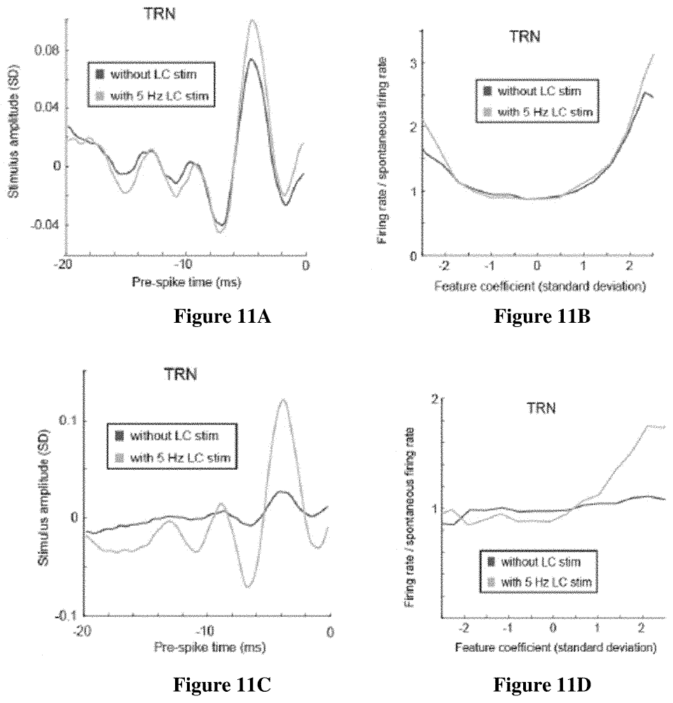

FIGS. 11A-11D show LC activation can (1) increase the rate of sensory related information transmitted for a subset of thalamic reticular nucleus (TRN) neurons and (2) induce gated feature selectivity in a subset of thalamic reticular nucleus (TRN) that did not selectively respond to features without LC stimulation.

Aspects described herein provide methods of modifying sensory processing in a subject, comprising applying continuous, tonic vagus nerve stimulation to a subject at a frequency of at least about 5 Hz. The term “continuous” refers to without interruption. The term “tonic” refers to a sustained or graded as compared to duty-cycled patterns. The term “modifying sensory processing” refers to changing sensory processing (e.g., vision, hearing, smell, taste, touch etc.) in a subject. In one aspect, the modification is improving sensory processing such that the subject performs tasks in an improved manner (e.g., faster, more accurate, for a longer period of time).

In another aspect, the amplitude of the continuous, tonic vagus nerve stimulation can be about 0.25 mA or from about 0.1 mA to about 3 mA.

The time for applying the continuous, tonic vagus nerve stimulation can be least at least about 1 seconds, 5 seconds, 10 seconds, 15 seconds, 30 seconds 45 second, 60 second, 90 seconds, 180 seconds or longer.

As discussed herein, the modified sensory processing occurs within less than about one second and is short term or transient (e.g., within about 1 minute following cessation of VNS).

Further aspects provide methods of modifying sensory processing in a subject, by: exposing the subject to a sensory stimulation; measuring a change in a pupil dilation from a first time point to a second time point; determining a mean value for the pupil dilation from the first time point to the second time point; measuring the pupil dilation and determining a pupil dilation value; and applying tonic, continuous vagus nerve stimulation to the subject when the pupil dilation value is at least two standard deviations from the mean value for pupil dilation.

In this aspect, pupil dilation can be measured with modified eyewear or a camera. For example, a subject (e.g., air traffic control personnel) can wear modified glasses (e.g., Google glass or similar device) that monitors pupil dilation during a series of tasks. Pupil dilation can be calibrated by calculating a mean for pupil dilation from a first time point to a second time point. Periodic measurements can be taken during a series of tasks. When pupil dilation is at least about two standard deviations from the mean value, vagus nerve stimulation can be applied as described herein for a desired period of time (e.g., 1, 5, 10, 15, 30, 45, 60, 90 seconds etc.) or continuously during a given task (e.g., guiding the landing of a plane).

In another aspect, pupil dilation or another proxy for reduced sensory processing can be measured by an algorithm or machine learning method to determine when VNS stimulation is needed and the length of time for VNS treatment.

Further aspects provide methods of modifying sensory processing in a subject, by detecting when the subject to a predetermined sensory stimulation; applying tonic, continuous vagus nerve stimulation to the subject when the predetermined sensory stimulation is detected; and discontinuing applying continuous vagus nerve stimulation to the subject when the predetermined sensory stimulation is not detected.

In this aspect, a subject can be exposed to a predetermine stimulus (e.g., photograph, document, human or animal, car on assembly line etc.) and vagus nerve stimulation (e.g., continuous, tonic vagus nerve stimulation) can be applied only when the predetermined stimulus is present and discontinued the predetermined stimulus is not present. For example, a subject operating a quality control inspection of a product can use this aspect to improve sensory processing only when the product being inspected is present. The presence or absence of an object can be determined using a camera or other sensory, smart eyewear etc.

Further aspects provide an apparatus for applying continuous, tonic vagus nerve stimulation to a subject in accordance with methods described herein. Such an apparatus can be operated manually by a subject in order to apply vagus nerve stimulation on demand. In another aspect, the device can further include a prosthetic device adapted to attach to a body part (i.e., arm, leg, head, torso etc.) and apply vagus nerve stimulation to improve sensory processing to accomplish a particular task.

Aspects described herein provide methods of modifying a sensory processing in a subject, comprising applying a tonic vagus nerve stimulation to the subject wherein a modifying of sensory processing comprises increasing a sensory acuity of the subject.

In some instances, the sensory processing is modified within less than about 1 second.

In some instances, the modified sensory processing is transient, and the effects of applying a tonic vagus nerve stimulation to the subject disappear within a minute of cessation of vagus nerve stimulation.

In some instances, the vagus nerve stimulation is continuous.

In some instances, the vagus nerve stimulation is discontinuous, and a time period of a portion of the discontinuous stimulation wherein vagus nerve stimulation is not applied is not greater than about 7 to about 10 seconds.

In some instances, a rate of sensory related information transmitted by a thalamocortical relay neuron in a subject is increased by at least about 100 to 200% compared to a subject that does not receive the vagus nerve stimulation.

In some instances, the modifying of sensory processing comprises improving a sensory perception in a subject having one or more impaired senses.

In some instances, the subject has impaired senses caused by a condition selected from the group consisting of aging, traumatic brain injury (TBI), neurological disorders, fatigue, inattention, and neurodegeneration.

Aspects described herein provide methods of modifying sensory processing in a subject, by detecting when the subject is in need of a sensory processing modification, applying tonic vagus nerve stimulation to the subject to provide the sensory processing modification and discontinuing applying the sensory processing modification when the subject no longer is in need of the sensory processing modification.

In some instances, detecting that the subject is in need of the sensory processing modification comprises measuring a change in a signal from a first time to a second time, determining a mean value and a variance value for the signal from the first time to the second time, determining a measured value for the signal, and applying tonic vagus nerve stimulation to the subject when the measured value is at least one to three standard deviations from the mean value.

In some instances, the signal being measured is selected from the group consisting of pupil diameter, EEG synchronization, relative power band strength, heart rate, heart rate variability, blood pressure, ECOG, respiratory rate, perspiration, skin conductivity, and signals recorded from invasive or noninvasive brain-machine interface.

In some instances, the vagus nerve stimulation is continuous.

In some instances, the vagus nerve stimulation is discontinuous , and wherein a time period of a portion of the discontinuous stimulation wherein vagus nerve stimulation is not applied is not greater than about 7 to about 10 seconds.

In some instances, a rate of sensory related information transmitted by a thalamocortical relay neuron in a subject is increased on average by at least about 100 to 200% compared to a subject that does not receive the vagus nerve stimulation.

In some instances, the modifying of sensory processing comprises improving a sensory perception in a subject having one or more impaired senses.

In some instances, the subject has impaired senses caused by a condition selected from the group consisting of aging, traumatic brain injury (TBI), neurological disorders, fatigue, inattention, and neurodegeneration.

Aspects described herein provide a vagus nerve stimulation device adapted to apply a tonic vagus nerve stimulation to a subject to modify sensory processing in the subject, wherein a modifying of sensory processing comprises increasing a sensory acuity and wherein a time of applying the tonic vagus nerve stimulation is at least about 4 minutes.

In one aspect, the modified sensory processing is transient, and the effects of applying a tonic vagus nerve stimulation to the subject disappear within a minute of cessation of vagus nerve stimulation.

In another aspect, the vagus nerve stimulation is continuous.

In a further aspect, the vagus nerve stimulation is discontinuous, and a time period of a portion of the discontinuous stimulation wherein vagus nerve stimulation is not applied is not greater than about 7 to about 10 seconds.

In one aspect, a rate of sensory related information transmitted by a thalamocortical relay neuron in a subject is increased on average by at least about 100 to 200% compared to a subject that does not receive the vagus nerve stimulation.

In another aspect, the device further comprises a prosthetic device adapted to be associated with a body part of the subject and wherein the prosthetic device is adapted to direct the vagus nerve stimulation to a cervical or auricular region of the subject.

In yet another aspect, the prosthetic device is selected from the group consisting of eyeglasses, sunglasses, a hearing aid, a neck brace, a craniofacial prosthetic, a voice prosthetic, compression stimulation devices, sensory neuroprostheses, an orbital prostheses, a cervical collar, a halo vest, a dental implant, a facial implant, a helmet, a vehicle or machinery cockpit, machinery controls, a head-up display, a headset, a necklace, earrings, goggles, a tiara, a scarf, jewelry, a headdress, a headscarf, a hat, a tie, a bonnet, ear muffs, headphones, headsets, a shawl, a lanyard, a wig, a hood, a headband, a hair tie, a beret, a hair clip, a neck pillow, a shirt collar, a rifle scope, binoculars, a night vision device, a telescope, virtual reality headset, a video game controller, a video game system, clothing, an adhesive patch, a blood pressure monitor, a heart rate monitor, an oximeter, a watch, a smart watch, a phone, and 3D glasses.

### EXAMPLES

### Example 1

To understand the extent to which VNS modulates thalamic sensory processing, single-unit activity was recorded from the VPm (ventral posteromedial nucleus) of the rat vibrissa pathway in response to repeated WGN whisker deflection while VNS stimulation patterns were systematically varied (FIG. 1A). The VPm is a relay nucleus in the thalamus that gates somatosensory information to cortex^{47,48}. VPm neurons reliably respond to stimulation of the neuron's corresponding principle whisker^{49, 50} (FIG. 1B). Four different VNS patterns were tested: no stimulation (as a control), standard duty-cycle (30 Hz, 30 s on/60 s off duty-cycle), continuous tonic (10 Hz), and fast duty-cycle (30 Hz, 3 s on/7 s off duty-cycle) (FIG. 1C). Each VNS pattern lasted 180 s, during which 12 repetitions of the frozen 15 s WGN whisker stimulation were delivered, with a at least 75 s of rest period between them.

### VNS Modulation of Sensory Processing is Transient

To ensure the system had ample time to reset to baseline conditions during the rest periods interleaved between VNS conditions, each VPm neuron's response during the control time period without VNS stimulation was compared to the same neurons response occurring during the second half of all of the rest periods (45-75 s after the cessation of the preceding VNS condition). Confirming correct experimental design, the effects of VNS on sensory processing were transient and dissipated within 60 seconds of cessation of VNS. This was quantitatively confirmed as there was no significant difference in feature modulation (FIG. 1D; 1 during control period vs 0.96±0.04 during second half of rest periods, 36 features, 25 neurons, 6 rats, p=0.27, paired t-test), the percent of spikes in bursts (FIG. 1E; 23±2% during control period vs 24±2% during second half of rest periods, 25 neurons, 6 rats, p=0.48, paired t-test), and information transmission (FIG. 1F; 0.13±0.03 bits/spike during control period vs 0.14±0.04 bits/spike during second half of rest periods, 36 features, 25 neurons, 6 rats, p=0.21, Wilcoxon signed-rank test).

These results suggest that, unlike previously reported VNS-induced effects which are neuroplasticity-based and last over long timescales, VNS enhancement of sensory processing rapidly dissipates following cessation of VNS. Further, this confirms that the periods of rest time inserted between VNS conditions in this experiment were long enough to allow for the system to return to baseline conditions.

### Example 2

### Standard Duty-Cycle VNS Improved Thalamic Feature Selectivity and Information Transmission

To estimate the feature selectivity of VPm neurons and the effects of VNS on thalamic sensory processing, the response of VPm neurons to the same frozen white Gaussian noise (WGN) whisker stimulation with and without VNS was compared. The striations clearly visible in the raster plots of recorded VPm spiking activity in response to repeated presentations of the same WGN stimulation indicated that the neurons were sensitive to certain kinetic features in the stimulus, as the cells reliably fired at certain time points during each presentation (FIG. 2A).

Standard duty-cycle VNS (i.e. 30 Hz, 30 s on/60 s off) did not change the firing rate of the thalamic relay neurons (FIG. 2B; 11.0±0.6 Hz during control periods vs 11.5±0.7 Hz during standard duty-cycle VNS, 25 neurons, 6 rats, p=0.20, paired t-test; Mean±SEM reported for all results unless otherwise stated). Spike triggered covariance analysis was used to assess the selectivity of the response of the VPm neurons to specific features^{1, 51} (FIG. 2C). This showed that VNS improved the feature selectivity of VPm neurons as indicated by (1) an increase in the amplitude of the recovered kinetic features the neurons selectively responded to, and (2) the tilting up of nonlinear tuning function at high feature coefficient values¹ (FIG. 2D).

As the magnitude of the feature coefficient at any given time point represents the similarity between the stimulus and a feature, this alteration in the shape of the nonlinear tuning function indicates an increased selectivity of the neuron to only spike-following stimuli that closely match the neuron's encoded feature. To quantitatively measure the change in the amplitude of the recovered features, a feature modulation factor as previously defined was used¹ (see Methods). A feature modulation factor of 1 suggests that there was no significant change in encoded kinetic features, whereas a value greater than 1 suggests an increase in amplitude without a change in shape. Standard duty-cycle VNS was found to result in feature modulation factors significantly larger than 1 (FIG. 2E; 1 without VNS vs 1.21±0.05 during standard duty-cycle VNS, 36 features, 25 neurons, 6 rats, p=1.8e-2, paired t-test).

To quantify the effects of VNS on both the encoded kinetic features and nonlinear tuning functions for each neuron, an information theoretic approach was employed to estimate the information transmitted by each VPm spike about the presence/absence of the encoded feature in the stimulus'. Consistent with observations of improved feature selectivity, standard duty-cycle VNS dramatically increased both information transmission efficiency (FIG. 2F; 202±27% of control bits/spike with standard duty-cycle VNS, 36 features, 25 neurons, 6 rats, p=5.0e-5, Wilcoxon signed-rank test; FIG. 21, 0.13±0.03 bits/spike without VNS vs 0.20±0.05 bits/spike with standard duty-cycle VNS, 36 features, 25 neurons, 6 rats, p=4.6e-4)) and information transmission rate (FIG. 2J; 206±28% of control bits/second with standard duty-cycle VNS, 36 features, 25 neurons, 6 rats, p=1.4e-6, Wilcoxon signed-rank test).

Consistent with previous work, thalamic relay neurons exhibited burst firing under control conditions^{1, 52}. Since thalamic bursts have been linked to deterioration of transmission of information about detailed stimulus features^{53,54}, VNS-induced enhancement of sensory processing might also coincide with suppressed burst firing of VPm neurons. Thalamic burst spikes did not transmit as much information as tonic spikes (FIG. 2G, 0.18±0.05 bits/spike with tonic spikes vs 0.035±0.005 bits/spike with burst spikes, without VNS, 36 features, 25 neurons, 6 rats, p=1.3e-4, Wilcoxon signed-rank test). When comparing the information transmitted by tonic spikes to that transmitted by each burst when considered as a point event, burst events on average transmitted less information than tonic spikes (FIG. 2G, 0.18±0.05 bits/spike with tonic spikes vs 0.080±0.01 bits/spike with burst events, without VNS, 36 features, 25 neurons, 6 rats, p=0.08, Wilcoxon signed-rank test). However, the difference was not quite significant, most likely due to limited sampling. As expected, VNS decreased the fraction of VPm spikes in bursts (FIG. 2H; 23±2% without VNS vs 21±2% during standard duty-cycle VNS, 25 neurons, 6 rats, p=1.3e-3, paired t-test).

### Example 3

The short timescale of VNS effects on thalamic sensory processing caused standard duty-cycle patterns of VNS to induce a fluctuating thalamic sensory processing state

A typical therapeutically employed VNS stimulation pattern traditionally uses a relatively slow duty-cycle (e.g. 30 s on/60 s off). The off period of the standard VNS pattern used described herein (60 s) is longer than the period it takes for the effects of VNS on sensory processing to dissipate (-45 s). Although relatively slow duty-cycled patterns have proved to efficiently mitigate symptoms in neurological disorders, it was unclear how switching VNS on and off would modulate thalamic state given that the effects of VNS on VPm sensory processing occur and dissipate on such short timescales.

To test this, the responses of VPm neurons during the on period of VNS were compared to the same neurons' responses during the first 30 s and second 30 s of the off period. Interestingly, the effect of VNS on thalamic feature selectivity and information transmission rapidly diminished during the off period. The amplitude of the recovered encoded features was significantly smaller during the second 30 s of the VNS off period than during the VNS on period (FIG. 3A). Quantifying this difference in recovered feature amplitude using the feature modulation factor, the factor was larger during the on sections than the off sections of the standard duty-cycle VNS (FIG. 3B; 1.20±0.06 during on period vs 1.06±0.05 during second half of off period, 36 features, 25 neurons, 6 rats, p 2.8e-2, paired t-test).

The fluctuations in thalamic processing state induced by standard duty-cycle VNS was further evidenced by the observation that there was a significant change in percent of spikes in bursts in the second 30 s of the VNS off period as compared to the VNS on period (FIG. 3C; 19±2% during on period vs 22±2% during second half of off period, 25 neurons, 6 rats, p=8.2e-5, paired t-test).

Accordingly, the information transmitted per spike was significantly less during the second half of the off period than the on period of the standard duty-cycle VNS (FIG. 3D; 254±31% of control bits/spike during on period vs 190±26% of control bits/spike during second half of off period, 36 features, 25 neurons, 6 rats, p=1.3e-2, paired t-test). Taken together these results indicate that standard duty-cycle VNS created a fluctuating state of sensory processing in the thalamus that is sub-optimal for perceptual sensitivity. This fluctuating state would be sub-optimal for perceptual sensitivity, as the same stimulus occurring during the on period of the VNS cycle would evoke a different thalamic response than if it occurred during the off period of the VNS cycle and therefore may be incorrectly perceived as a different stimulus.

### Example 4

Tonic and Fast duty-cycle VNS (i.e. 3 sec on/7 sec off) enhanced thalamic information transmission without inducing fluctuations

In this example, the data suggests that VNS rapidly induces improvement in thalamic sensory processing, and that this improvement quickly fades away once VNS is turned off. In addition, the data suggests that standard duty-cycle VNS patterns create a fluctuating sensory processing state. As described herein, one way to achieve the benefits of VNS on sensory processing without creating a fluctuating processing state would be to use fast duty-cycle VNS (e.g. 3 s on/7 s off) or continuous tonic VNS which do not have long off periods. To assess whether these stimulation patterns could be used for optimal, fluctuation-free enhancement of sensory processing, standard duty-cycle (30 son 60 s off), fast duty-cycle (3 s on 7 s off), and continuous (10 Hz) VNS were performed in the same recording session, and the effects of the various VNS patterns on thalamic feature selectivity were compared. None of standard duty-cycle (30 s on 60 s off), fast duty-cycle (3 s on 7 s off), and continuous (10 Hz) VNS resulted in a significantly different VPm firing rate as compared to control conditions (FIG. 4A; 11.0±0.6 Hz without VNS vs 10.9±0.7 Hz during 10 Hz tonic VNS, 11.2±0.7 Hz during fast duty-cycle VNS, and 11.6±0.7 Hz during standard duty-cycle VNS, 25 neurons, 6 rats, p=0.79, 0.53 and 0.21 respectively, paired t-test).

Standard duty-cycle (30 s on 60 s off), fast duty-cycle (3 s on 7 s off), and continuous (10 Hz) VNS produced similar improvements in (1) thalamic feature selectivity as quantified by the feature modulation factor (FIG. 4B; 1.12±0.05 during standard duty-cycle VNS vs 1.14±0.04 during 10 Hz tonic VNS or 1.15±0.05 during fast duty-cycle VNS, 36 features, 25 neurons, 6 rats, p=0.61 and 0.33, respectively, paired t-test) and (2) information transmission efficiency (FIG. 4C; 202±27% of control bits/spike during standard duty-cycle VNS vs 197±19% of control bits/spike during 10 Hz tonic VNS or 223±29% of control bits/spike during fast duty-cycle VNS, 36 features, 25 neurons, 6 rats, p=0.84 and 0.19, respectively, paired t-test; FIG. 4E 0.20±0.05 bits/spike during standard duty-cycle VNS vs 0.18±0.04 bits/spike during 10 Hz tonic VNS and 0.20±0.05 bits/spike during fast duty-cycle VNS, 36 features, 25 neurons, 6 rats, p=0.77 and 0.53 respectively, Wilcoxon signed-rank test and paired t-test respectively).

Standard duty-cycle (30 s on 60 s off), fast duty-cycle (3 s on 7 s off), and continuous (10 Hz) VNS produced a VPm response with a similar percent of spikes in bursts with all VNS patterns resulting in a decrease in the percent of spikes in bursts when compared to control conditions. (FIG. 4D; 21±2% during standard duty-cycle VNS vs 20±2% during 10 Hz tonic VNS or 21±2% during fast duty-cycle VNS, 25 neurons, 6 rats, p=0.04 and 0.56, respectively, paired t-test). To investigate whether fast duty-cycle VNS introduced any fluctuations in VPm sensory processing state similar to those observed to be induced by standard duty-cycle VNS (in a similar fashion as to the analysis of the different stages of the standard duty-cycle) the response of the VPm neurons during the on periods of the fast duty-cycle stimulus was segmented, and compared with the same neuron's response during the first or second half of the off period.

Here, there was no significant difference in firing rate (FIG. 4F; 11.3±0.7 Hz during on period vs 11.2±0.7 Hz during first half of off period or 11.1±0.7 Hz during second half of off period, 25 neurons, 6 rats, p=0.19 and=0.22 respectively, paired t-test) and percent of spikes in bursts (FIG. 4G; 21±2% during on period vs 21±2% during first half of off period or 21±2% during second half of off period, 25 neurons, 6 rats, p=0.59 and=0.85 respectively, paired t-test) between on the on period of fast duty-cycle VNS and the first half or second half of the off cycle.

Both the improvement in feature selectivity and change in nonlinear tuning function did not fluctuate between the on period and first half and second half of the off periods of fast duty-cycle VNS. This lack of fluctuation in feature selectivity during fast duty-cycle translated to no difference in the feature modulation factor between the on period and either half of the off period (FIG. 4H; 1.12±0.05 during on period vs 1.18±0.06 during first half of off period or 1.17±0.07 during second half of off period, 36 features, 25 neurons, 6 rats, p=0.30 and=0.37 respectively, paired t-test).

Further, there was no difference in the strength of improvement of information transmission efficiency between the on period and either half of the off periods of fast duty-cycle VNS (FIG. 41; 236±32% of control bits/spike during on period vs 223±25% of control bits/spike during first half of off period or 256±45% of control bits/spike during second half of off period, 36 features, 25 neurons, 6 rats, p=0.64 and=0.89 respectively, paired t-test and Wilcoxon signed-rank test respectively).

Together, these exemplary results indicate that both fast duty-cycle VNS and tonic VNS result in the same level of improvement in thalamic sensory processing as standard duty-cycle VNS, without inducing a fluctuating thalamic sensory processing state that was induced by standard duty-cycle VNS. This is important as during a fluctuating thalamic sensory processing state, the same stimulus would evoke a different thalamic response if received at different time points in the fluctuation which may degrade the ability to discriminate between similar stimuli.

### Example 5

The Effects of Fast Duty-Cycle and Tonic VNS on Thalamic Sensory Processing were Amplitude Dependent

These results indicate that both fast duty-cycle and tonic VNS can be used to optimally enhance thalamic sensory processing whereas standard duty-cycle VNS is suboptimal for this purpose as it induces fluctuations in thalamic processing state. During the experiments which compared the effects of these stimulation patterns, all VNS pulses were delivered at a fixed current amplitude of 1 mA. However, The amplitude of VNS being currently used in clinical situations can vary from patient to patient and exists within a wide range of values⁵⁵⁻⁵⁷. It has been found that some effects of VNS have an inverted U shape relationship with VNS amplitude⁵⁸⁻⁶². Therefore, to determine the effects of different amplitudes of VNS on sensory processing, new experiments were conducted to examine the sensitivity of VNS effects on thalamic information transmission to VNS amplitude. Four different VNS amplitudes were compared: 0 (as a control), 0.4 mA, 1 mA, and 1.6 mA.

When analyzing fast duty-cycle VNS at different amplitudes, none of the three amplitudes induced changes in VPm firing rate in response to WGN whisker stimulation as compared to the control period (FIG. 5A, 11.3±2.1 Hz during control without VNS vs. 11.6±2.4 Hz during 0.4 mA fast duty-cycle VNS, 11.1±2.3 Hz during 1 mA fast duty-cycle VNS, and 10.36±1.8 Hz during 1.6 mA fast duty-cycle VNS, 7 neurons, 2 rats, p =0.65, 0.80, and 0.21 respectively, paired t-test).

Fast duty-cycle VNS-induced improvement in feature selectivity and information transmission monotonically increased with amplitude (FIG. 5B) as quantitatively measured by the feature modulation factor (FIG. 5C; 1 during control without VNS vs. 0.98±0.07 during 0.4 mA fast duty-cycle VNS, 1.05±0.07 during 1 mA fast duty-cycle VNS, or 1.11±0.04 during 1.6 mA fast duty-cycle VNS, 13 features, 7 neurons, 2 rats, p=0.78, 0.44, and 0.02 respectively, paired t-test) and information transmission efficiency (FIG. 5D, 116±12% of control bits/spike during 0.4 mA fast duty-cycle VNS, 138±14% of control bits/spike during 1 mA fast duty-cycle VNS, or 144±17% of control bits/spike during 1.6 mA fast duty-cycle VNS, 13 features, 7 neurons, 2 rats, p=0.20, 1.6e-2, and 2.3e-2 respectively, paired t-test).

Burst firing also deceased monotonically with the increase in fast duty-cycle VNS amplitude as evidenced by a decrease in the percent of spikes in bursts (FIG. 5E, 14.9±2.4% during control without VNS vs. 13.5±2.3% during 0.4 mA fast duty-cycle VNS, 11.5±2.0% during 1 mA fast duty-cycle VNS, or 11.3±2.0% during 1.6 mA fast duty-cycle VNS, 7 neurons, 2 rats, p=0.17, 1.6e-2, and 6.9e-4 respectively, paired t-test).

Similarly, when analyzing 10 Hz tonic VNS at different amplitudes, none of the three amplitudes induced changes in VPm firing rate in response to WGN whisker stimulation as compared to the control period (FIG. 5F, 10.0±1.1 Hz during control without VNS vs. 9.9±1.1 Hz during 0.4 mA 10 Hz VNS, 9.4±1.1 Hz during 1 mA 10 Hz VNS, and 9.1±1.1 Hz during 1.6 mA 10 Hz VNS, 16 neurons, 5 rats, p=0.84, 0.46, and 0.08 respectively, paired t-test).

Tonic VNS-induced improvement of feature selectivity monotonically increased with amplitude of VNS (FIG. 5G) as quantitatively measured by feature modulation factor (FIG. 5H; 1 during control without VNS vs. 0.95±0.05 during 0.4 mA 10 Hz VNS, 1.12±0.06 during 1 mA 10 Hz VNS, or 1.28±0.06 during 1.6 mA 10 Hz VNS, 24 features, 16 neurons, 5 rats, p=0.33, 0.048, and 2.0e-4 respectively, paired t-test) and information transmission efficiency (FIG. 51, 125±8% of control bits/spike during 0.4 mA 10 Hz VNS, 182±17%of control bits/spike during 1 mA 10 Hz VNS, or 272±38% of control bits/spike during 1.6 mA 10 Hz VNS, 24 features, 16 neurons, 5 rats, p=7.5e-3, 7.4e-5, and 1.7e-4 respectively, paired t-test).

Burst firing also decreased monotonically with the increase in tonic VNS amplitude as evidenced by a decrease in the percent of spikes in bursts (FIG. 5J, 28.1±3.3% during control without VNS vs. 27.1±3.5% during 0.4 mA 10 Hz VNS, 24.7±3.4% during 1 mA 10 Hz VNS, or 22.5±3.3% during 1.6 mA 10 Hz VNS, 16 neurons, 5 rats, p=0.36, 5.6e-2, and 6.9e-5 respectively, paired t-test).

Taken together, these characterization results suggest that VNS rapidly improves thalamic sensory processing in an amplitude dependent fashion.

### Example 6

The Effects of VNS on Thalamic Sensory Processing were Frequency Dependent

VNS with different frequencies can have distinguishable effects in clinical applications⁵⁵⁻⁵⁷. Therefore, it was important to evaluate how different frequencies of VNS affect thalamic sensory processing. To this end the responses of VPm neurons during 10 Hz, 1 mA continuous tonic VNS were compared to the same neurons' responses during 30 Hz, 1 mA continuous tonic VNS stimulation (taken from the On periods of the standard duty-cycle VNS).

Again, both frequencies of tonic VNS resulted in firing rates that were not significantly different than during the control period (FIG. 6A; 11.0±0.6 Hz during control without VNS vs 10.9±0.7 Hz with 10 Hz VNS or 11.3±0.7 Hz during 30 Hz VNS, 25 neurons, 6 rats, p=0.79 and 0.49 respectively, paired t-test).

Percent of spikes in bursts decreased monotonically with increasing tonic VNS frequency (FIG. 6B; 23.0±2.3% during control without VNS vs 19.4±2.2% with 10 Hz VNS or 18.8±2.0% during 30 Hz VNS, 25 neurons, 6 rats, p=1.2e-5 and 1.8e-5 respectively, paired t-test).

Moreover, 30 Hz VNS produced a stronger increase in recovered feature amplitude and tilting up of the nonlinear tuning function. When the effects of 10 Hz and 30 Hz VNS on the recovered features were quantified, it was observed that both produced a significantly larger feature modulation factor than 1, which increased monotonically with increasing tonic VNS frequency (FIG. 6C, 1 during control without VNS vs 1.14±0.04 during 10 Hz VNS or 1.20±0.06 during 30 Hz VNS, 36 features, 25 neurons, 6 rats, p=1.7e-3 and 1.9e-3 respectively, paired t-test).

Consequently, due to VNS effects on sensory processing increasing monotonically with tonic VNS frequency, the information transmission efficiency also monotonically increased with VNS frequency (FIG. 6D, 198±19% of control bits/spike during 10 Hz VNS vs. 255±32% of control bits/spike during 30 Hz VNS, 36 features, 25 neurons, 6 rats, p=8.2e-6 and 2.2e-5, respectively, paired t-test). Information transmission efficiency was significantly more strongly improved with 30 Hz VNS than with 10 Hz (FIG. 6E, p=6.8e-3, paired t-test).

### Example 7

### LC Modulation of Thalamoreticulo-Thalamic Circuit Dynamics Changed the Temporal Structure Used by VPm Neurons to Encode the Same WGN Whisker Stimulus.

Single-unit activity of VPm neurons in response to repeated presentations of a frozen WGN whisker deflection pattern was recorded while activation condition of the LC-NE system in pentobarbital-anesthetized rats was varied⁶³. Here, the encoding of the high dimensional spatiotemporal whisker deflection signal into a neuron's spike train was modeled using the linear-nonlinear-Poisson cascade model^{51, 64}.

In response to multiple presentations of the same WGN whisker stimulation, VPm neurons respond reliably at specific timepoints which correspond to sections of the stimulus which closely match the kinetic features the neuron selectively encodes for. These timepoints at which a reliable response occurs, called events, were identified through using a threshold (3× mean firing rate) to identify peaks in the spike density function (SDF). Once multiple responses of a neuron to the same frozen stimulus have been recorded, the SDF was generated by first collapsing the perievent raster into a peristimulus time histogram (PSTH), then smoothing the PSTH by convolving it with an adaptive kernel (see methods).

Through reverse correlation analysis, the kinetic feature(s) to which each VPm neuron selectively responded to was recovered and then the corresponding nonlinear tuning function(s) was calculated, illustrating the sensitivity of the neuron's response to how closely the stimulus resembles that feature. An information theoretic approach was used (see methods) to quantify the mutual information between a neuron's spike response and the absence/presence of the features the neuron selectivity encodes for in the stimulus⁶³.

Interestingly, previous work showed that neither an LC-induced general reduction in firing rate nor the LC-activation-induced improvement in reliability could be responsible for the observed LC-activation-induced improvement in information transmission efficiency and rate⁶³. Further, previous work had also found that removal of bursting spikes could also not explain the observed LC-activation-induced enhancement of sensory processing, as tonic spikes during LC activation carried significantly more information than tonic spikes without LC activation⁶³. How then is the temporal pattern of the VPm response used to encode the absence/presence of features in the incoming stimulus changed in such a way as to optimize the efficiency and rate of the information transmitted?

To investigate this question further the temporal structure of events each VPm neurons used to encode the frozen WGN stimulus with and without LC stimulation was compared. When the perievent raster and SDF of the VPm response with 5 Hz LC stimulation was overlayed over that of the VPm response without LC stimulation, it can be clearly seen that LC stimulation alters the temporal event structure (FIG. 7A).

Some events are conserved across both control and LC-activation conditions (FIG. 7A, conserved events labeled with purple boxes). However, LC-activation results in the removal of some events that were present under controlled conditions (FIG. 7A, removed events labeled with red boxes). Further, LC-activation results in the addition of some new events that were not present under control conditions (FIG. 7A, emerged events labeled with green boxes).

This suggest that LC-activation may optimize the temporal structure each VPm neuron uses to encode a specific stimulus by removing less-optimal events and adding more-optimal events.

To allow for further analysis of the change in temporal event structure, event types were classified as follows. Any 5 Hz LC stimulation events that overlapped with a 0 Hz LC stimulation event were considered “conserved events”. VPm events during 0 Hz LC stimulation which did not overlap with any events during 5 Hz LC stimulation were considered “removed events” while VPm events during 5 Hz LC stimulation which did not overlap with any events during 0 Hz LC stimulation were considered “emerged events” (FIG. 7A).

Here it was found that approximately half of the events found during control conditions were removed with LC stimulation; while approximately 40 percent of the events found during 5 Hz LC-activation were newly emerged and not present during control conditions (FIGS. 7B-7C, 32 neurons across 19 rats).

### Example 8

### LC-activation Resulted in a Removal of Less Informative Events and an Introduction of More Informative Events

Next, to determine if there was any difference in the feature selectivity of the spikes in the different event types, four different groups of spikes were selected: spikes without LC stimulation that occurred during removed events, spikes without LC stimulation that occurred during conserved events, spikes during 5 Hz LC-activation that occurred during conserved events, and spikes during 5 HZ LC-activation that occurred during emerged events.

When the feature selectivity for spikes in each subtype of event is recovered, the feature selectivity of spikes during LC-activation that fell within newly emerged events had an improved feature selectivity as compared to spikes during control conditions that fell within removed events (FIGS. 8A-8B). This improved feature selectivity of emerged vs removed events indicates the LC-mediated removal and introduction of events favors optimal feature selectivity.

The amplitude of the recovered features for removed and emerged event spikes was then compared with that of the amplitude of the feature selectivity recovered using all control condition spikes by calculating the feature modulation factor (see methods). The feature modulation factor increases to values greater than 1 when the recovered feature amplitude is greater than that of the feature selectivity recovered during the control periods. Here, indeed spikes during emerged events had a significantly greater feature modulation factor than spikes during removed events (FIG. 8C, 1.0±0.1 for spikes within removed events vs 1.7±0.1 for spikes within emerged events, 59 features across 32 neurons across 19 rats, p=6.2e-5, paired t-test).

An information theoretic approach was then employed to quantify the information transmitted by these spikes about the absence/presence of the feature they selectively encode for in the stimulus. The results showed that spikes within emerged events carried significantly more information than spikes within removed events (FIG. 8D, 0.20±0.02 bits/spike within removed events vs 0.67±0.10 bits/spike within emerged events, 59 features across 32 neurons across 19 rats, p=3.9e-9, Wilcoxon signed-rank test).

When comparing spikes that occurred during conserved event times without LC stimulation with spikes that occurred during conserved event times with LC stimulation, it was found that the feature selectivity of these spikes are improved by LC-activation as well (FIGS. 8A-8B). This suggests that even within conserved events, LC-activation causes a shift of the distribution of spikes into the more informative conserved events and away from the less informative conserved events.

A significantly greater feature modulation factor for spikes that occurred during conserved event times with LC stimulation than spikes that occurred during conserved event times without LC stimulation was observed (FIG. 8E, 1.8±0.1 for spikes within conserved events without LC stimulation vs 2.2±0.1 for spikes within conserved events with 5 Hz LC stimulation, 59 features across 32 neurons across 19 rats, p=1.3e-6, paired t-test).

Further, spikes that occurred during conserved event times with LC stimulation carried significantly more information than spikes that occurred during conserved event times without LC stimulation (FIG. 8F, 0.37±0.05 bits/spike within conserved events without LC stimulation vs 0.93±0.14 bits/spike within conserved events with 5 Hz LC stimulation, 59 features across 32 neurons across 19 rats, p=9.3e-10, paired t-test).

To verify that the change in event structure and observed differences in the information encoded by spikes within the different event types was not an artifact of the threshold chosen to identify event times from the SDF, the above analysis was performed again, but using different event thresholds (e.g. 2× and 4× mean firing rate).

With both of these new event thresholds there is still the same increase in bits/spike for emerged vs removed events (FIG. 8G, 2× mean firing rate event threshold, 0.15±0.02 bits/spike within removed events without LC stimulation vs 0.69±0.14 bits/spike within emerged events with 5 Hz LC stimulation, 59 features across 32 neurons across 19 rats, p=6.8e-9, Wilcoxon signed-rank test, FIG. 8I, 4× mean firing rate event threshold, 0.29±0.04 bits/spike within removed events without LC stimulation vs 0.81±0.14 bits/spike within emerged events with 5 Hz LC stimulation, 59 features across 32 neurons across 19 rats, p=8.5e-11, Wilcoxon signed-rank test).

Further with both event thresholds there is still the same increase in bits/spike between spikes in conserved events occurring with LC stimulation vs spikes in conserved events occurring without LC stimulation (FIG. 8H, 2× mean firing rate event threshold, 0.25±0.03 bits/spike within conserved events without LC stimulation vs 0.82±0.12 bits/spike within conserved events with 5 Hz LC stimulation, 59 features across 32 neurons across 19 rats, p=7.1e-9, Wilcoxon signed-rank test, FIG. 8J, 4× mean firing rate event threshold, 0.45±0.06 bits/spike within conserved events without LC stimulation vs 1.07±0.16 bits/spike within conserved events with 5 Hz LC stimulation, 59 features across 32 neurons across 19 rats, p=1.7e-9, Wilcoxon signed-rank test).

### Example 9

### The Reorganization of the Temporal Response Events of VPm Neurons During LC Activation Favors Ideal Event Placement for Feature Selectivity

Having found that LC-activation results in a restructuring of the temporal positions of the reliable response events used by the same VPm neuron to encode the same stimulus, how ideal the encoding pattern of each VPm neuron was with and without LC stimulation was investigated. In this example, a definition of what an ideal encoding of the stimulus into a corresponding response events would look like for a neuron with a specific feature selectivity was determined.

Here, the search is constrained for each neuron's ideal response by using the same exact number of events in the ideal response as were present in each neuron's actual SDF.

To find ideal the timepoints to place these events, first the feature coefficient value was calculated (i.e. the dot product between the 20 ms of preceding stimulus and feature selectivity) for each timepoint of the WGN stimulus (FIG. 9A-9B).

Without being bound by theory, it is believed that a very informative neuron would only respond at the timepoints when the feature coefficient has a large magnitude (e.g. the peaks in the resulting feature coefficient vector). However, whether a neuron's response is directionally sensitive to the sign of the feature coefficient (i.e. sensitive to only large positive feature coefficient values vs large negative and positive feature coefficient values) varies across neurons. A neuron selectively responding to a specific feature in a directional fashion would ideally fire at large magnitude feature coefficients only if they are positive value (FIG. 9A). While a neuron selectively responding to a specific feature in a non-directional fashion would ideally fire at large magnitudes of feature coefficients regardless of whether they were negative (the inverse of the feature) or positive (FIG. 9B).

To determine whether a neuron's feature selectivity was directional or non-directional, for each feature the directionality of the corresponding nonlinear tuning index was quantified using an directionality alpha value as defined by⁵¹ (see methods). A feature selectivity which is directionally selective will exhibit an asymmetric nonlinear tuning function (FIG. 9A, right panel), and will have an alpha value close to 1. A feature selectivity which is not directionally selective will have a corresponding nonlinear tuning function that appears symmetric across the y axis (FIG. 9B, right panel), and an alpha value close to 0.

Interestingly, LC stimulation slightly increased the directionality of VPm feature selectivity as measured by alpha (FIG. 9D, alpha=0.44±0.04 without LC stimulation vs 0.55±0.04 with 5 Hz LC stimulation, 59 features across 32 neurons across 19 rats, p=6.8e-4, paired t-test).

When deciding if each feature a neuron selectively responded to was encoded in a directional manner or not, the average directionality alpha values between the feature selectivity with and without LC-activation was used. Any resulting average directionality alpha value which fell beneath a threshold (alpha=0.3) was considered to be non-directionally selective while any average that fell above was considered to be directionally selective.

After the directionality of each feature selectivity was calculated then the peaks in the corresponding feature coefficient vector which would be most ideal to position our events could be identified.

Here, the same number of events as observed in the original response was conserved but the event times were moved to be ideally located, i.e. at the peaks in the feature coefficient vector with the largest positive values for directionally selective features (FIG. 9A, red stars) or largest absolute values for non-directionally selective features (FIG. 9B, red stars).

The encoding by these ideal event timepoints was than compared with the actual event timepoints observed with and without LC stimulation (FIG. 9A-9B, blue stars). Here LC stimulation increased the fraction of events that occurred at an ideal event timepoint (FIG. 9C, 0.20±0.01 without LC stimulation vs 0.23±0.01 with 5 Hz LC stimulation, 59 features across 32 neurons across 19 rats, p=6.0e-6, paired t-test).

Taken together with the previous results, this shows that LC-activation results in a changing of the temporal event structure in such as a way that favors more informative, and therefore more optimal, event locations.

### Example 10

Thalamicortical Responses During LC Activation Can Be Decoded Into a More Accurate Reconstruction of the Original Stimulus than without LC Activation

Next, to investigate how the changes in the stimulus encoding properties of individual thalamicortical neurons impacted the ability of a population of VPm neurons to accurately encode a spatiotemporal whisker stimulus, a subset of VPm recordings was selected for which all the responses where driven by the same frozen WGN stimulus. From an ideal observer standpoint of view, it was analyzed as to how accurately decoding and reconstructing the original stimulus knowing only the VPm neurons' responses and feature selectivity could be done with and without LC stimulation (see Methods).

To reconstruct the original stimulus, it was assumed the preceding strength of the feature in the stimulus was relative to the average spiking response of the neuron at that timepoint (See Methods). Initially, only directional feature selectivity was used to reconstruct an approximation as non-directional feature selectivity needed to be orientated correctly to improve reconstruction accuracy.

The reconstruction was then improved using non-directional feature selectivity by assuming the direction of the feature selectivity at any timepoint is equal to that of the approximation reconstructed using only directional feature selectivity.

Interestingly, the final reconstruction is more accurate when using the spiking response and feature selectivity of the neurons during 5 Hz LC stimulation as compared to the reconstruction generated using the spiking response and feature selectivity of the neurons without LC stimulation (FIG. 10A).

This shows that LC stimulation optimizes the encoding of sensory-related information in the thalamus in a manner which allows for a more accurate recovery of the original stimuli from the thalamocortical spike trains, suggesting the accuracy of the perception of stimuli could be enhanced as well. Indeed, in previous work, it was found that LC-stimulation enhanced the perceptual sensitivity of rats discriminating between two different frequencies of whisker stimulation⁶³.

To quantify how LC stimulation affects how closely the reconstruction matches the original stimulus, and how LC stimulation affected the ratio of decoded stimulus accuracy to features used to decode it, the above method of decoding of the stimulus from directional PSTH-feature pairs multiple times was performed multiple times for each possible number of features used. For each directional reconstruction, the correlation coefficient between that reconstruction and the original stimulus was saved.

When looking at a plot of average correlation coefficient versus number of features used for directional reconstruction, it was found that the accuracy of the reconstruction increases with increasing number of features used (FIG. 10B).

It was also found that adding another feature reduced accuracy, indicating there is some redundancy in the information carried by each feature selectivity.

In this example, no matter how many features are used for reconstruction, LC stimulation resulted in a more accurate reconstruction as measured by either correlation coefficient (FIG. 10B) or RMSE between the reconstruction and original stimulus (FIG. 10C).

Interestingly, when reconstructing with directional features only, the difference between the accuracy of reconstruction with and without LC stimulation increased as the number of features decoded from increases. Without being bound by theory, it is believed that perhaps LC activation decreases the redundancy of information carried by VPm neurons.

A similar analysis investigating how the accuracy of the directional reconstruction is improved by adding in different amounts of non-directional features improved the reconstruction was then performed (FIG. 10B). The results of this analysis also showed that LC stimulation results in a more accurate reconstruction when decoded from both directional and non-directional features (FIG. 10B, FIG. 10C).

### Example 11

### LC Stimulation Affected the Feature Selectivity of a Subset of TRN Neurons.

Here, the effects of LC activation on TRN feature selectivity were elucidated. Interestingly, approximately 43 percent of TRN neurons exhibited a significant feature selectivity with and without LC stimulation.

Of these TRN neurons, which always exhibit feature selectivity, approximately half of them exhibited an LC-activation-induced improvement in feature selectivity (FIG. 11A-11B).

20 percent of TRN neurons did not exhibit a significant feature selectivity without LC stimulation, but did have a significant feature selectivity with 5 Hz LC stimulation (FIG. 11C-11D). The feature selectivity of these neurons could then be considered gated by LC activation, only occurring during states of high arousal as indexed by LC activity.

This suggest that LC-activation induced changes in intrathalamic dynamics allow for TRN neurons to respond to whisker stimuli in a more feature selective manner.

If TRN neurons project to, and therefore inhibit, VPm neurons with relatively orthogonal feature selectivity, then increases in the selectivity of the TRN neurons can sharpen the innervated VPm neurons' feature selectivity. For example, inhibitory TRN neurons selectively responding to features relatively orthogonal to the feature selectivity of the VPm neuron which they inhibit will result in an inhibition of the VPm neuron's response at timepoints when the stimulus does not closely match the innervated VPm neuron's feature selectivity. A shift from general to feature selective TRN inhibition of VPm neurons may explain why LC-activation changes the temporal response structure of a VPm neuron to the same whisker stimulus.

### Discussion

Previous work has focused on using VNS to facilitate the neuroplasticity of brain circuits, likely through activation of neuromodulatory systems which are known to induce neuroplasticity⁶⁵. These changes require pairing stimuli or tasks with VNS activation and take place over weeks to months²⁰. In contrast, as described herein, it was found that VNS was also able to drastically affect the sensory processing within the thalamus at a short timescale, requiring no prior pairing. Further, the effects of VNS on sensory processing were found to be transient as they dissipated quickly following cessation of VNS. This new application of VNS therefore does not depend on long-term changes induced by neuroplasticity, instead VNS activation results in rapid, transient regulation of sensory processing in the thalamus most likely through activation of neuromodulation centers that can rapidly change thalamic neurochemical state, such as the LC.

VNS-induced improvements of thalamic sensory processing occurred through enhancement of feature selectivity and resulted in an increased efficiency and rate of sensory information transmitted by the VPm neurons. Previous studies have shown a causal link between enhanced thalamic sensory processing and improved perceptual performance^{1, 66}. Therefore, as this data shows that VNS improves thalamic sensory processing, it suggests that certain patterns of VNS could potentially be used to improve behavioral performance in perceptual tasks.

VNS improved thalamic feature selectivity and information transmission in similar fashion as direct LC stimulation.

Previous work demonstrated a causal relationship between LC-stimulation induced suppression of thalamic bursts and improvement in information transmission¹, it is important to note that VNS also suppressed burst firing in the thalamus. This is not unexpected as it has been shown that the vagus nerve exerts influence on LC activity through the projection of the NTS and that VNS increases LC activity^{6, 67}.

However, the NTS also projects to neuromodulatory nuclei other than the LC, including the basal forebrain⁶⁸ which projects to the sensory thalamus as well. Activation of either the LC or the basal forebrain has been shown to modulate sensory processing^{1, 69, 70}. Therefore, the improved thalamic sensory processing observed here may be attributed to the collective action of the modulatory systems activated by VNS. It is worth noting that neuromodulatory nuclei are heavily interconnected^{71, 72}. For example, VNS has been shown to exert excitatory influence on both the LC and the dorsal raphe nucleus but there is no direct projection from the NTS to the dorsal raphe nucleus^{6, 73}. Therefore, VNS may modulate thalamic sensory processing through either direct or indirect activation of the different neuromodulatory systems.

Previous work using the cat common peroneal nerve model has shown that neural tissue is less likely to be damaged when using electrical stimulation delivered with an intermittent duty-cycle⁷⁴. In current clinical treatments, VNS is most commonly given in a duty-cycle fashion, such as 30 s on/60 s off^{55-57, 75}, which is based on the assumption that duty-cycled stimulation poses less of a risk of damaging a nerve⁷⁴. VNS improvement of thalamic sensory processing is transient and rapidly dissipates following cessation of VNS, which resulted in the effects of VNS dissipating during the off periods of the standard duty-cycle VNS. This fluctuating thalamic processing state resulted in VPm neurons exhibiting a difference in feature modulation, sensory information transmission efficiency, and burst firing rate during the on versus the off period of standard duty-cycle VNS.

Standard duty-cycle VNS-induced fluctuating sensory processing state would presumably induce a fluctuating bias in perception that was not related to the stimulus and therefore would act as noise, therefore it is particularly detrimental to the precise information processing needed during perceptual discrimination tasks. For example, the same stimulus would produce different neural responses if received during the on period versus the off period of the standard duty-cycle, which may cause the same stimuli to be perceived as two different stimuli.

VNS with a fast duty-cycle of 3 s on 7 s off did not induce fluctuations in thalamic sensory processing state, presumably due to the fact that the time constants of VNS modulation of sensory processing in the thalamus are faster than those of standard duty-cycle VNS patterns but not those of a fast duty-cycle VNS pattern.

Increasing the frequency of VNS as well as the amplitude of fast duty-cycle VNS and tonic VNS resulted in stronger improvements in sensory processing as evidenced by increased feature selectivity and improved stimulus-related information transmission. These results suggest that an optimal state for perceptual processing is best achieved using high frequency and high amplitude VNS delivered either continuously or at least with a high frequency duty-cycle.

Strong types of VNS patterns potentially pose a higher risk of vagus nerve damage or patient discomfort if delivered too aggressively. One method effectively enhance perception of stimuli with minimal nerve damage risk would be to time the activation of the continuous tonic VNS relative to the stimulus events, so that tonic VNS is delivered continuously during any time period which the user might receive a behaviorally important stimulus but is shut off in between these periods when stimuli will not be received. This type of stimulus-locked VNS-enhancement of sensory processing would be facilitated by the fact that VNS-induced improvements in perception rapidly onset once VNS is initiated. Previous work suggested that the activation of the LC-NE system is more beneficial during harder tasks'. Therefore, task-dependent on-demand VNS may be an optimal configuration in enhancing behavioral performance.

The ability to switch on fast duty-cycle or tonic VNS during time periods when sensory perception enhancement is required, would be highly helpful for individuals suffering from these disorders. For example, individuals with compromised senses of touch often struggle with tasks such as buttoning their shirt or grasping objects³⁰. A non-invasive VNS system could be designed in such a way that the user could turn it on prior to the task and switch it off afterwards. This would allow for a high frequency, high amplitude, continuous tonic or fast duty-cycle VNS without risk of nerve damage as the time period in use would be relatively minimal. This type of on-demand perception enhancement device is possible due to the rapid onset of sensory processing enhancement by VNS shown here, and does not require long-term periods of stimulation to see effects such as the neuroplasticity-based methods used to treat epilepsy and depression.

Newly developed sensory neuroprotheses have attempted to use patterned microstimulation of different regions along the sensory pathway, such as the sensory cortex and thalamus, to recover senses lost due to disease, degeneration, or injury⁷⁶⁻⁸¹. When using these neuroprotheses to write information to the brain to produce desired perception, the state of the brain regions being written to can be taken into consideration as brain state heavily influences perception and behavior^{82, 83}. Changes in brain state may cause the same microstimulation pattern to produce different results of neuron activation or may change the reading-out of the resulting neuron activation by higher-order brain regions and therefore cause the same microstimulation pattern to evoke different perceptual experiences.

This study suggests the increases in feature selectivity and improvements in information transmission that result from LC activation and VNS occur due to a shift in intrathalamic dynamics that reduces membrane potential fluctuations of sensory thalamic relay neurons¹. Membrane potential fluctuations are non-optimal for sensory processing as they introduce a non-stimulus-related bias. For this same reason it is likely that membrane potential fluctuations would be non-optimal for the writing of information to sensory regions necessitated by sensory neuroprotheses. Therefore, coupling the ability to modulate information processing state through patterned VNS with current sensory neuroprosthetic writing techniques, may allow for improvements in the accuracy and reliability of neuroprosthetic sensations.

Tailoring brain-state to create an optimal state for writing information to the brain could also be applicable to non-invasive brain stimulation methods for sensory and cognitive neuroprotheses as fluctuating brain state would induce the same bias on their ability to reliably and accurately write information to regions along the pathway.

In sensory neuroprostheses using patterned microstimulation, a major goal is for patients to better discriminate between sensations evoked by microstimulation from neighboring electrodes in the implanted array. For example, the ability of individuals with cochlear implants to discriminate between stimulation from neighboring electrodes in their cochlear implant varies widely across patients⁸⁴ and improved discrim inability is associated with better speech recognition. Therefore, it would be worthwhile to investigate the effects of non-invasive-VNS-induced modulation on the minimal distance between discriminable microstimulation electrodes in sensory systems.

LC tonic activity has been correlated with sensory processing, with increased tonic firing causing improved sensory processing and perceptual discrimination abilities. Causal links between LC tonic activity and pupil size and cortical EEG pattern have also been shown 21, indicating that the LC activity can be indexed using changes in pupil diameter and/or EEG patterns. Therefore, a self-optimizing sensory enhancement neuroprothesis could consist of a closed loop system. This system would read out the current state of arousal and sensory processing via tracking pupil diameter and/or other physiological signals indexing brain state. It could then identify time periods in which the user's sensory processing is drifting away from detailed, feature identification and discrimination to more basic detection and correct this change by delivering VNS.

Previous research has suggested that certain pharmaceutical compounds, such as amphetamines, may enhance processing of sensory stimuli⁸⁵⁻⁸⁷. VNS enhancement of sensory processing could either replace or augment pharmacological treatments. VNS is superior to pharmaceuticals as non-invasive VNS does not suffer from tolerance build up associated with pharmacological techniques and can be tuned to have minimal side effects⁸⁸.

Previous work has shown that increases in thalamic feature selectivity and information transmission efficiency and rate translate to improved performance on perceptual discrimination tasks 1. Specifically, the ability of rats to discriminate between whisker stimuli delivered at varying frequencies was improved as evidenced by an increased perceptual sensitivity (d′). Results described herein suggest that non-invasive VNS can be used to rapidly enhance perceptual sensitivity in humans. This could be helpful for many work-related tasks such as image and audio discrimination or operating machinery. Further, VNS-induced enhancement of sensory processing could be beneficial for military personnel and sports and e-sports, where the ability to discriminate between small differences in visual, auditory, and tactile stimuli make a huge difference on performance.

Further supporting our argument that LC enhances sensory processing not primarily through modulation of gain or SNR (signal to noise ratio), it was unexpectedly found that LC activation changes the temporally precise firing pattern the VPm uses to encode the same stimulus. However, it is counterintuitive that this change in encoding pattern would occur without a change in the kinetic features for which the neuron is encoded. Using the methods and devices described herein, the new encoded pattern is optimized as it increases the efficiency and rate of stimulus-related information transmitted by VPm neurons.

Previously, LC-induced enhancement of sensory processing was shown to result in an enhancement of the feature selectivity as well as an improvement of information transmission efficiency and rate of VPm neurons⁶³. Here, it was shown that LC stimulation allows for a more accurate recovery of the original stimulus when decoding it from the response of a population of VPm neurons as an ideal observer, suggesting that LC stimulation enhances the accuracy of the perception of whisker stimuli.

When investigating whether event timepoints occur at ideal locations, a VPm neuron may selectively encode for multiple features. Therefore, event timepoints which may be non-ideal for one of the neuron's feature may be ideal for another. Interestingly, as described herein, an increase in the fraction of events occurring at ideal times for the feature selectivity of neurons selective for one feature as well as neurons selective for multiple. If the change in the temporal structure of reliable events used to encode a whisker stimulus resulted in an improved feature selectivity for one feature at the cost of a degraded feature selectivity for another feature one would expect to see a mixed result of LC activation on the fraction of events at ideal times. Instead, observed improvement occurred across the vast majority of feature selectivity, suggesting that removed events were not ideal events for any of the features the neuron encoded for. In this way LC-mediated change in temporal response structure does not shift the feature selectivity towards one feature at the expense of another, but rather improves the feature selectivity for all features selectively responded to by the neuron.

As shown previously, the mechanism underlying this optimization of thalamic state for sensory processing is the action of LC-induced increased NE concentration in the thalamus. The action of NE resulted in a reduction in calcium t-channel activity in both the VPm and TRN, which is believed to decrease the subthreshold membrane potential fluctuations of VPm neurons. Removal of these underlying noisy fluctuations may change the response of VPm neurons to be more solely related to stimulus-relevant input from the PrV.

It has been proposed that sensory processing exists along a gradient between a bursting thalamic state optimized for detection and a tonic thalamic state optimized for discrimination⁸⁹⁻⁹². The TRN receives topographically aligned input from sensory thalamus regions, and in return provides topographically aligned inhibitory input to thalamic relay cells^{93,94}, thus whether the TRN is responding in a non-selective bursting fashion or a tonic feature selective manner heavily impacts thalamocortical transmission of sensory information.

During times of high LC arousal, when a stimulus is delivered to the whisker, VPm neurons whose feature selectivity most closely matches the incoming stimulus would likely spike first, and through the negative feedback loop of the TRN may inhibit the response of other competing VPm neurons whose feature selectivity less closely matches the stimulus, as their response would be predicted to be relatively delayed. Therefore, in this state the selective TRN inhibitory feedback creates a winner-takes-all response in which only the VPm neurons whose feature selectivity most closely matches the stimulus are given the opportunity to spike. This type of encoding would enhance the discrim inability of stimuli as different stimuli would evoke unique populations of VPm neurons.

During states of low LC arousal, calcium t-channels are likely to be primed and therefore a whisker stimulus is likely to evoke a rapid response from multiple VPm neurons, even those whose feature selectivity doesn't closely match the stimulus, as their response is facilitated by the all-or-nothing nature of calcium t-channel activation. In this state, the TRN selective feedback would not occur quickly enough to beat out the VPm action potentials due to calcium t-channel activity boosting the rate at which those action potentials occur. Instead the TRN feedback would be received after the VPm neurons have already spiked, which would then push the VPm neurons further into a hyperpolarized state and therefore re-prime their calcium t-channels for bursting activity. This type of encoding would enhance the detection of stimuli, as every stimulus would evoke a strong multi-neuron response, but would degrade the discrimination of different stimuli as they may evoke population responses that are too overlapping to discriminate

Although the aspects described herein analyzed the tactile sensory pathway, it is believed that the LC-NE system modulates sensory processing of visual and auditory modalities in a similar manner. This is because previous research has correlated increased attention and NE levels with reduced bursting activity in both visual and auditory thalamocortical neurons^{89, 90, 95-101}. As the LC is a well-known neuromodulator of attention and arousal¹⁰², these findings indicate the LC is able to optimize perception in a behavioral-state-relevant manner^{92, 103} by improving thalamocortical transmission of detailed sensory information during time periods of increased attention and arousal. Therefore, methods and devices herein can be used for LC modulation gustatory and olfactory sensory processing as well.

It is important to note that neuromodulatory systems are well persevered over evolution, and the function of neuromodulatory systems are similar in humans and other mammals such as rodents¹⁰⁴, Therefore, the results of these preclinical studies are translatable to humans as shown by the well-accepted animal models used herein. Methods

Surgery. All animal work was approved by the Columbia University Institutional Animal Care and Use Committee and the procedures were conducted in compliance with NIH guidelines. 16 adult albino rats (Sprague-Dawley, Charles River Laboratories, Wilmington, Mass.; ˜225-275 g at time of implantation) were used in this study. Animals were housed 1-2 per cage in a dedicated housing facility, which maintained a twelve-hour light and dark cycle.

Rats were sedated with 5% vaporized isoflurane in their home cages before being transported to the surgery suite at 2% vaporized isoflurane. Rats where then mounted on a stereotaxic frame, and the anesthetic was switched to ketamine/xylazine (80/8 mg/kg)⁶. Body temperature was kept at 37° C. by a servo-controlled heating pad (FHC Inc, Bowdoin, Me.). Blood-oxygen saturation level and heart rate were continuously monitored using a non-invasive monitor (Nonin Medical Inc, Plymouth, MN).

To allow for implantation of the VNS cuff, an incision was made on the left ventral side of the rats. A magnetic fixator retraction system (Fine Scientific Tools, Foster City, Calif.) was used to separate the sternohyoid and sternomastoid muscles longitudinally, providing clear access to the vagus nerve running next to the carotid artery within the carotid sheath. Glass tools were used to separate the vagus nerve from the carotid sheath so as to minimize any potential damage to the nerve. A platinum-iridium bipolar cuff electrode¹⁰⁵ was then placed around the vagus nerve to allow for delivery of VNS. An insulated lead connected to the VNS cuff was then ran out of the incision, which was closed with sutures.

Following VNS implantation, the animal was carefully mounted on a custom-modified stereotaxic frame (RWD Life Science, China) on top of a floating air table to allow for a craniotomy to be created above the VPm to for insertion of a recording electrode. Any exposed brain surface was then covered in warm saline, contained by retaining wells created around the craniotomies.

Electrophysiology. Single, sharp, tungsten microelectrodes (75 pm in diameter, impedance of ˜3-5 MΩ, FHC Inc, Bowdoin, Me.) were used to record extracellular single-unit activity. A hydraulic micropositioner (David Kopf, Tujunga, Calif.) allowed for slow, controlled electrode positioning with micrometer resolution, and thus allowed for close proximity placement to recorded neurons. Extracellular neural signals were referenced to a ground screw in contact with the surface of the dura, contralateral to the recording site, then band-pass filtered (300-8k Hz) and digitized at 40 kHz using a Plexon recording system (OmniPlex, Plexon Inc., Dallas, Tex.). Spike sorting of single units was performed using commercially available software (Offline Sorter, Plexon).

The VPm was targeted using stereotaxic coordinates from the rat brain atlas 106. VPm neuron identity was confirmed by a strong response to the mechanical stimulation of the neuron's principal whisker⁴⁸⁻⁵⁰. Only large, easily isolatable VPm units with a minimum refractory period greater than 1 ms and a stable waveform throughout the entire recording were used. Burst spiking was defined as any two or more spikes occurring with an ISIs (interspike intervals) of 4 ms or less and following at least 100 ms of quiescence⁵³.

Vagus Nerve Stimulation. The vagus nerve cuff lead was connected to a calibrated electrical microstimulator (Multi Channel Systems, Reutlingen, Germany), which was then triggered by an xPC target real-time system (MathWorks, Mass.) running at 1 kHz. During periods of VNS, cathode-leading biphasic current pulses (250 ps per phase) were delivered at either 10 or 30 Hz with amplitudes of either 0.4, 1, or 1.6 mA with duty-cycles of either continuous, fast (3 s on/7 s off), or standard (30 s on/60 s off). For each recording, multiple repetitions of each VNS condition were delivered in a random order. Each VNS condition delivery lasted 180 s with 75-90 seconds of rest time inserted following to allow for the system to reset to baseline conditions before beginning the next condition. As currently practiced in humans, only the left vagus nerve was stimulated as stimulation of the right vagus nerve has been shown to cause cardiac irregularities due to right vagus nerve efferents innervating the sinoatrial node¹⁰⁷. Further, the polarity of VNS was fixed, with the (negative electrode cranial) as a reversal of this polarity has been shown to induce bradycardia¹⁰⁸.

Whisker Stimulation. A custom modified galvo motor (galvanometer optical scanner model 6210H, Cambridge Technologies) controlled by a closed-loop system (micromax 67145 board, Cambridge Technology) as described in¹⁰⁹ was used to deliver precise, high-frequency mechanical whisker stimulations (12.5 mm shaft). The galvo motor's position was controlled via the same xPC target real-time system controlling VNS/LC activation. Accuracy of whisker stimulation was verified by using the Plexon recording system to also record the galvo motor's output analog position signal. Whiskers were cut to a length of ˜10 mm and inserted into the deflecting arm, which was positioned ˜5 mm from whiskerpad. The WGN was low pass filtered (butterworth, 10th order) at 250 Hz¹. The galvo motor was used to continuously deliver whisker deflection following a signal consisting of continuous repetitions of a 15 second clip of frozen white Gaussian noise (WGN). The plane of whisker deflection was fixed throughout the recording to determine if neurons had similar or altered responses to identical stimuli under varying conditions of VNS.

Data Analysis. Here, it was assumed that VPm neurons encode for stimulus-related information via the linear-nonlinear-Poisson model (LNP) as previously detailed by^{1, 51, 64}. Through analyzing the neuron's spiking response to a repeated delivery of a frozen WGN whisker deflection pattern, the neurons' feature selectivity can be recovered, which can be represented by a linear filter set and the corresponding set of nonlinear tuning functions. Specifically, each neuron's first significant feature was recovered as the spike triggered average (STA) whisker displacement during the 20 ms window preceding each spike. Spike triggered covariance (STC) analysis was then used to recover the remaining set of significant features for any neurons which selectively responded to more than one kinetic feature⁶⁴.

$$S T A = \frac{1}{N} {\sum }_{n = 1}^{N} \vec{S} ( {t}_{n} )$$

$$S T C = \frac{1}{N - 1} {\sum }_{n = 1}^{N} {[ \vec{S} ( {t}_{n} ) - S T A ] [ \vec{S} ( {t}_{n} ) - S T A ]}^{T}$$

Where tₙ is the time of the n^{th} spike, \vec{S}(tₙ) is a vector representing the stimulus during the temporal window preceding a spike, and N is the total number of spikes.

Statistical significance of STAs was determined using a bootstrap procedure with 1000 bootstrap trials. Recovered STAs were considered insignificant if their amplitude fell within the 99.9 percentile of the bootstrap displacement range. The significance of STC recovered filters was determined using nestled bootstrapping of the eigenvalues corresponding to the STC recovered filters. A recovered eigenvalue that exceeded the 99.9 percentile of its corresponding bootstrap range of its filter was considered significant. Neurons without significant feature selectivity were excluded from further analysis.

To quantify the modulation of the recovered features by LC activation, a feature modulation factor is defined as¹:

$$feature modulation factor = \frac{control feature \cdot conditional feature}{control feature \cdot control feature}$$

To estimate each nonlinear tuning function corresponding to each significant recovered feature, the feature coefficient for each spike (i.e. the dot product between a neuron's linear filter and the stimulus preceding each spike) was calculated. The probability distribution of feature coefficient values k given a spike (i.e. Prob(k|spike)) could then be determined. To calculate all possible feature coefficients for the stimulus used, a 20 ms window was slid through the 15 s WGN stimulus, from which a probability distribution of all feature coefficient values (i.e. Prob(k)) was generated. By dividing Prob(klspike) by Prob(k), the nonlinear tuning functions are produced that map firing rate to feature coefficient value.

To quantify the information the spike train conveys about the absence/presence of a feature under varying VNS or LC stimulation conditions, mutual information between the presence/absence of a feature and the observation of a spike for each condition was calculated as¹¹⁰

$$Info ( k ; spike ) = \int dk * Prob ( k \vert spike ) * {log}_{2} ( \frac{Prob ( k \vert spike )}{Prob ( k )} )$$

Where k is the feature. Information transmission rate (i.e. bits/second) was calculated by multiplying bits/spike by the average firing rate of the neuron in response to WGN stimulus.

Statistics. A one-sample Kolmogorov-Smirnov test was used to assess the normality of data before performing statistical tests. If the samples were normally distributed, a paired or unpaired t-test was used. Otherwise, the Mann-Whitney U-test was used for unpaired samples or the Wilcoxon signed-rank test for paired samples. Multiple comparisons were corrected with Bonferroni correction.

Electrophysiology. All experimental data analyzed in this study were previously published in a study investigating how LC activation affects thalamic feature selectivity⁶³. Detailed surgical and electrophysiological methods behind the generation of the data can be found detailed in⁶³. Briefly, rats where anesthetized with sodium pentobarbital and mounted to a stereotaxic frame to allow for craniotomies to be performed which gave access to the LC and VPm or TRN. For rats which underwent electronic LC microstimulation, a recording electrode was advanced into the LC, with LC location being confirmed by the characteristic response of LC neurons to paw pinch¹¹¹. The recording system was then disconnected, and the electrode was connected to an electrical microstimulator (S88, Grass Instrument, Warwick, R.I.). For rats that underwent optogenetic LC stimulation, 4 weeks prior to the experiment, a lentivirus was injected directly into the rat's LC which allowed for selective transfection of noradrenergic neurons to express Channelrhodopsin2 (pLenti-PRSx8-hChR2(H134R)-mCherry, the UNC vector core, ˜7e9 vp/ml). At the beginning of an optogenetic LC stimulation experiments, a fiber optic cannula was advanced so as to be positioned against the LC, and then was attached to an LED driver (Plexon, 493 nm wavelength). For all experiments, a recording electrode was then advanced into the VPm or TRN, with VPm/TRN neurons being identified by their stereotaxic coordinates and response to punctate whisker deflection⁴⁹.

Experimental paradigm. The experimental procedures briefly described here are discussed in more detail in the original publication of this data set⁶³. Briefly, a frozen block of WGN whisker deflection was repeatedly delivered to the primary whisker via a custom modified galvomotor¹⁰⁹ (galvanometer optical scanner model 6210H, Cambridge Technologies) controlled by a closed-loop system (micromax 67145 board, Cambridge Technology). Single-unit recordings of VPm neurons responses to multiple repetitions of the stimulus were then captured via a Plexon recording system (OmniPlex, Plexon Inc., Dallas, Tex.). During each recording, LC activation condition was varied. Therefore, at the end of each recording a data set of multiple responses of the same VPm neuron to the same frozen WGN stimulus for each condition of LC activation was generated. This allowed us to analyze how LC activation changes the way in which each VPm neuron selectively encodes for information about the kinetic features in the stimulus. In one aspect, the response of VPm neurons without LC stimulation versus their response with 5 Hz LC stimulation was analyzed.

Reverse correlation analysis. The response of both VPm and TRN neurons using the linear-nonlinear-Poisson cascade model (LNP) was modeled^{51, 64}. Through analyzing multiple responses of a neuron to the same frozen WGN stimulus, the kinetic features can be identified in the stimulus to which the neuron selectively responds. Each neuron's significant features were recovered by first calculating the spike triggered average (STA) followed by calculating the spike triggered covariance (STC) matrix to recover the remaining set of significant features for any neurons which selectively responded to more than one kinetic feature^{51, 64}.

$$S T A = \frac{1}{N} {\sum }_{n = 1}^{N} \vec{S} ( {t}_{n} )$$

$$S T C = \frac{1}{N - 1} {\sum }_{n = 1}^{N} {[ \vec{S} ( {t}_{n} ) - S T A ] [ \vec{S} ( {t}_{n} ) - S T A ]}^{T}$$

Where tₙ is the time of the n^{th} spike, \vec{S}(tₙ) is a vector representing the stimulus during the temporal window preceding that spike, and N is the total number of spikes. Statistical significance of features was determined using bootstrap procedures⁶⁴. To quantify the change in amplitude of features recovered during different LC activation conditions, a feature modulation factor was used, previously defined as⁶³:

$$feature modulation factor = \frac{control feature \cdot conditional feature}{control feature \cdot control feature}$$

Once the linear portion of the LNP model was recovered, i.e. the kinetic features the neuron selectively responded to, the corresponding nonlinear tuning functions for each feature can be calculated by dividing the probability distribution of feature coefficients given a spike by the probability distribution of all possible feature coefficients found in the stimulus:

$$Nonlinear tuning function = \frac{Prob ( k \vert spike )}{Prob ( k )} .$$

Where k is feature coefficient values, i.e. the dot product between the linear filter and the preceding stimulus.

The strength of the directionality of the selective response to a specific feature was quantified via analyzing the symmetry of the nonlinear tuning function as follows:

$$directionality alpha value = \frac{G ( B ) - G ( - B )}{G ( B )}$$

Where G is the nonlinear tuning function and B is equal to 2 standard deviations of feature coefficient value.

Information conveyed by VPm neurons about the features they selectively responded to was quantified as^{51, 110}:

$$Info ( k ; spike ) = \int dk * Prob ( k \vert spike ) * {log}_{2} ( \frac{Prob ( k \vert spike )}{Prob ( k )} )$$

Where k is the feature coefficient and the resulting bits/spike value indicates the mutual information between the absence/presence of that kinetic feature in the stimulus and the occurrence of a spike by this neuron.

To allow for identification of reliable events in the response of neurons to the same WGN whisker stimulus, the peristimulus time histogram (PSTH) of the neuron's responses was binned (2 ms bins) and convolved with an adaptive boxcar kernel¹¹², whose size was dynamically increased from 1 at each bin until the bins spanned by that kernel contained at least 10 spikes, to produce a spike density function (SDF). A threshold (3 times the mean firing rate unless otherwise stated) was then used to identify peaks in the SDF which were then considered events¹¹².

Decoding VPm responses. To reconstruct an approximation of the original stimulus from an ideal observer viewpoint the average temporal response pattern of each neuron to the incoming stimulus (e.g. the peristimulus time histogram) was calculated, as well as the features for which that neuron was encoded. For neurons that were selective for multiple features, each feature-PSTH pair was considered unique. Only the directionally selective feature-PSTH pairs were selected to use for the initial reconstruction. This was done as from an ideal observer viewpoint the non-directionally selective features are not informative until directionality of the stimulus can be predetermined.

For each directionally selective feature-PSTH pair, at each timepoint in the PSTH the preceding strength of the feature in the stimulus was assumed to be relative to the PSTH value in that bin (i.e. average spike count/trial at that timepoint). The reconstructed vector at each point for a directionally selective feature-PSTH pair was therefore calculated as:

$${reconstruction}_{directional feature} ( t ) = {\sum }_{i = 1}^{T - 1} feature ( T - i ) * P S T H ( t + i )$$

Where the bin size for both the PSTH and feature are equal to the sampling frequency of the original stimulus (i.e. 5000 Hz, 0.2 ms bins) and T is the length of the feature. All reconstruction vectors corresponding to each directional feature-PSTH pair were summed, and the z-score of the resulting vector to generate a reconstruction of the original stimulus was determined.

$$directional reconstruction = z score ( \sum {reconstruction}_{directional feature} )$$

Using the directional reconstruction to approximate the original stimulus direction at any timepoint, the reconstruction was further improved using the non-directionally selective feature-PSTN pairs. To this end, for each non-directionally selective feature-PSTN pair a reconstruction was generated which was at each point equal to:

$${reconstruction}_{non - directional feature} ( t ) = {\sum }_{i = 1}^{T - 1} A * feature ( T - i ) * PSTH ( t + 1 )$$

$$A = 1 if dot ( directional reconstruction ( t - T \text{:} t ) , feature ) \geq 0$$

$$A = - 1 if dot ( directional reconstruction ( t - T : t ) , feature ) < 0$$

The value of A effectively flips the feature at any timepoint so that its direction is chosen to be the direction which best matches the reconstruction generated from directional features only. Once a reconstructed stimulus vector was calculated for each non-directionally selective feature-PSTN pair, a reconstruction of the stimulus using both directional and non-directional feature-PSTN pair reconstructions was generated as:

$$complete reconstruction = z score ( \sum {reconstructions}_{directional feature} + \sum {reconstructions}_{non \text{-} directional feature} )$$

Statistics. All statistical tests were two-sided. A one-sample Kolmogorov-Smirnov test was used to assess the normality of data before performing statistical tests. If the samples were normally distributed, a paired or unpaired t-test was used. Otherwise, the two-sided Mann-Whitney U-test was used for unpaired samples or the two-sided Wilcoxon signed-rank test for paired samples. Bonferroni correction was used for multiple comparisons.

### REFERENCES

1. Rodenkirch, C., Liu, Y., Schriver, B. J. & Wang, Q. Locus coeruleus activation enhances thalamic feature selectivity via norepinephrine regulation of intrathalamic circuit dynamics. Nature Neuroscience 22, 120-133 (2019).
2. Aston-Jones, G. & Cohen, J. D. An integrative theory of locus coeruleus-norepinephrine function: adaptive gain and optimal performance. Annual Review of Neuroscience 28, 403-450 (2005).
3. Sara, S. J. The locus coeruleus and noradrenergic modulation of cognition. Nature Reviews Neuroscience 10, 211-223 (2009).
4. Joshi, S., Li, Y., Kalwani, Rishi M. & Gold, Joshua I. Relationships between Pupil Diameter and Neuronal Activity in the Locus Coeruleus, Colliculi, and Cingulate Cortex. Neuron 89, 221-234 (2016).
5. Günter, C., Delbeke, J. & Ortiz-Catalan, M. Safety of long-term electrical peripheral nerve stimulation: review of the state of the art. J Neuroeng Rehabil 16, 13-13 (2019).
6. Hulsey, D. R., et al. Parametric characterization of neural activity in the locus coeruleus in response to vagus nerve stimulation. Experimental neurology 289, 21-30 (2017).
7. Conway, C. R., Udaiyar, A. & Schachter, S. C. Neurostimulation for depression in epilepsy. Epilepsy & behavior: E&B 88s, 25-32 (2018).
8. Engineer, N. D., et al. Targeted Vagus Nerve Stimulation for Rehabilitation After Stroke. Frontiers in neuroscience 13, 280 (2019).
9. Peter, N. & Kleinjung, T. Neuromodulation for tinnitus treatment: an overview of invasive and non-invasive techniques. Journal of Zhejiang University. Science. B 20, 116-130 (2019).
10. Spindler, P., Bohlmann, K., Straub, H. B., Vajkoczy, P. & Schneider, U. C. Effects of vagus nerve stimulation on symptoms of depression in patients with difficult-to-treat epilepsy. Seizure 69, 77-79 (2019).
11. van Hoorn, A., et al. Neuromodulation of autism spectrum disorders using vagal nerve stimulation. Journal of clinical neuroscience : official journal of the Neurosurgical Society of Australasia 63, 8-12 (2019).
12. Yang, J. & Phi, J. H. The Present and Future of Vagus Nerve Stimulation. Journal of Korean Neurosurgical Society 62, 344-352 (2019).
13. Lamb, D. G., Porges, E. C., Lewis, G. F. & Williamson, J. B. Non-invasive Vagal Nerve Stimulation Effects on Hyperarousal and Autonomic State in Patients with Posttraumatic Stress Disorder and History of Mild Traumatic Brain Injury: Preliminary Evidence. Frontiers in medicine 4, 124 (2017).
14. Mwamburi, M., Liebler, E. J. & Tenaglia, A. T. Review of non-invasive vagus nerve stimulation (gammaCore): efficacy, safety, potential impact on comorbidities, and economic burden for episodic and chronic cluster headache. The American journal of managed care 23, S317-s325 (2017).
15. Reuter, U., McClure, C., Liebler, E. & Pozo-Rosich, P. Non-invasive neuromodulation for migraine and cluster headache: a systematic review of clinical trials. Journal of neurology, neurosurgery, and psychiatry (2019).
16. Hamer, H.M. & Bauer, S. Lessons learned from transcutaneous vagus nerve stimulation (tVNS). Epilepsy research 153, 83-84 (2019).
17. Mourdoukoutas, A. P., Truong, D. Q., Adair, D. K., Simon, B. J. & Bikson, M. High-Resolution Multi-Scale Computational Model for Non-Invasive Cervical Vagus Nerve Stimulation. Neuromodulation : journal of the International Neuromodulation Society 21, 261-268 (2018).
18. Hulsey, D. R., et al. Reorganization of Motor Cortex by Vagus Nerve Stimulation Requires Cholinergic Innervation. Brain Stimulation 9, 174-181 (2016).
19. Hays, S. A., Rennaker, R. L. & Kilgard, M. P. Targeting plasticity with vagus nerve stimulation to treat neurological disease. Prog Brain Res 207, 275-299 (2013).
20. Buell, E. P., et al. Vagus Nerve Stimulation Rate and Duration Determine whether Sensory Pairing Produces Neural Plasticity. Neuroscience 406, 290-299 (2019).
21. Liu, Y., Rodenkirch, C., Moskowitz, N., Schriver, B. & Wang, Q. Dynamic Lateralization of Pupil Dilation Evoked by Locus Coeruleus Activation Results from Sympathetic, Not Parasympathetic, Contributions. Cell reports 20, 3099-3112 (2017).
22. Correia, C., et al. Global Sensory Impairment in Older Adults in the United States. J Am Geriatr Soc 64, 306-313 (2016).
23. Heine, C. & Browning, C. J. Communication and psychosocial consequences of sensory loss in older adults: overview and rehabilitation directions. Disabil Rehabil 24, 763-773 (2002).
24. Serafini, G., et al. Extreme sensory processing patterns show a complex association with depression, and impulsivity, alexithymia, and hopelessness. Journal of affective disorders 210, 249-257 (2017).
25. Shimizu, V. T., Bueno, O. F. & Miranda, M. C. Sensory processing abilities of children with ADHD. Brazilian journal of physical therapy 18, 343-352 (2014).
26. Carron, S. F., Alwis, D. S. & Rajan, R. Traumatic Brain Injury and Neuronal Functionality Changes in Sensory Cortex. Frontiers in systems neuroscience 10, 47 (2016).
27. Al Saif, A. A. & Al Senany, S. Determine the effect of neck muscle fatigue on dynamic visual acuity in healthy young adults. J Phys Ther Sci 27, 259-263 (2015).
28. Anton-Erxleben, K. & Carrasco, M. Attentional enhancement of spatial resolution: linking behavioural and neurophysiological evidence. Nature reviews. Neuroscience 14, 188-200 (2013).
29. Tseng, Y. C., Liu, S. H., Lou, M. F. & Huang, G. S. Quality of life in older adults with sensory impairments: a systematic review. Qual Life Res 27, 1957-1971 (2018).
30. Nowak, D. A. & Hermsdorfer, J. Selective deficits of grip force control during object manipulation in patients with reduced sensibility of the grasping digits. Neuroscience research 47, 65-72 (2003).
31. Reed-Jones, R. J., et al. Vision and falls: A multidisciplinary review of the contributions of visual impairment to falls among older adults. Maturitas 75, 22-28 (2013).
32. Hennissen, L., et al. Cardiovascular Effects of Stimulant and Non-Stimulant Medication for Children and Adolescents with ADHD: A Systematic Review and Meta-Analysis of Trials of Methylphenidate, Amphetamines and Atomoxetine. CNS Drugs 31, 199-215 (2017).
33. Spiller, H. A., Hays, H. L. & Aleguas, A., Jr. Overdose of drugs for attention-deficit hyperactivity disorder: clinical presentation, mechanisms of toxicity, and management. CNS drugs 27, 531-543 (2013).
34. Dubljevic, V. Prohibition or coffee shops: regulation of amphetamine and methylphenidate for enhancement use by healthy adults. The American journal of bioethics: AJOB 13, 23-33 (2013).
35. Talih, F. & Ajaltouni, J. Probable Nootropicinduced Psychiatric Adverse Effects: A Series of Four Cases. Innovations in clinical neuroscience 12, 21-25 (2015).
36. Gualtieri, F. Unifi nootropics from the lab to the web: a story of academic (and industrial) shortcomings. Journal of enzyme inhibition and medicinal chemistry 31, 187-194 (2016).
37. Kilgard, M. P., Rennaker, R. L., Alexander, J. & Dawson, J. Vagus nerve stimulation paired with tactile training improved sensory function in a chronic stroke patient. NeuroRehabilitation 42, 159-165 (2018).
38. Engineer, C. T., et al. Temporal plasticity in auditory cortex improves neural discrimination of speech sounds. Brain Stimul 10, 543-552 (2017).
39. Borland, M. S., et al. Cortical Map Plasticity as a Function of Vagus Nerve Stimulation Intensity. Brain Stimul 9, 117-123 (2016).
40. Engineer, C. T., Engineer, N. D., Riley, J. R., Seale, J. D. & Kilgard, M. P. Pairing Speech Sounds With Vagus Nerve Stimulation Drives Stimulus-specific Cortical Plasticity. Brain Stimul 8, 637-644 (2015).
41. Darrow, M. J., et al. Restoration of Somatosensory Function by Pairing Vagus Nerve Stimulation with Tactile Rehabilitation. Annals of neurology 87, 194-205 (2020).
42. Borland, M. S., et al. Pairing vagus nerve stimulation with tones drives plasticity across the auditory pathway. Journal of neurophysiology 122, 659-671 (2019).
43. Loerwald, K. W., et al. Varying Stimulation Parameters to Improve Cortical Plasticity Generated by VNS-tone Pairing. Neuroscience 388, 239-247 (2018).
44. Meyers, E. C., et al. Enhancing plasticity in central networks improves motor and sensory recovery after nerve damage. Nature Communications 10, 5782 (2019).
45. Engineer, C., Hays, S. & Kilgard, M. Vagus nerve stimulation as a potential adjuvant to behavioral therapy for autism and other neurodevelopmental disorders. Journal of Neurodevelopmental Disorders 9 (2017).
46. Engineer, C. T., Engineer, N. D., Riley, J. R., Seale, J. D. & Kilgard, M. P. Pairing Speech Sounds With Vagus Nerve Stimulation Drives Stimulus-specific Cortical Plasticity. Brain Stimul 8, 637-644 (2015).
47. Diamond, M. E., von Heimendahl, M., Knutsen, P. M., Kleinfeld, D. & Ahissar, E. ‘Where’ and ‘what’ in the whisker sensorimotor system. Nature Reviews Neuroscience 9, 601-612 (2008).
48. Millard, D. C., Wang, Q., Gollnick, C. A. & Stanley, G. B. System identification of the nonlinear dynamics in the thalamocortical circuit in response to patterned thalamic microstimulation in vivo. Journal of Neural Engineering 10, 066011 (2013).
49. Wang, Q., Webber, R. & Stanley, G. B. Thalamic Synchrony and the Adaptive Gating of Information Flow to Cortex. Nature Neuroscience 13, 1534-1541 (2010).
50. Wang, Q., Millard, D. C., Zheng, H. J. V. & Stanley, G. B. Voltage-sensitive dye imaging reveals improved topographic activation of cortex in response to manipulation of thalamic microstimulation parameters. Journal of Neural Engineering 9, 026008 (2012).
51. Petersen, R. S., et al. Diverse and Temporally Precise Kinetic Feature Selectivity in the VPm Thalamic Nucleus. Neuron 60, 890-903 (2008).
52. Sherman, S. M. Dual response modes in lateral geniculate neurons: mechanisms and functions. Vis Neurosci 13, 205-213 (1996).
53. Sherman, S. M. Tonic and burst firing: dual modes of thalamocortical relay. Trends in Neurosciences 24, 122-126 (2001).
54. Wolfart, J., Debay, D., Le Masson, G., Destexhe, A. & Bal, T. Synaptic background activity controls spike transfer from thalamus to cortex. Nature Neuroscience 8, 1760 (2005).
55. Heck, C., Helmers, S. L. & DeGiorgio, C. M. Vagus nerve stimulation therapy, epilepsy, and device parameters: scientific basis and recommendations for use. Neurology 59, S31-37 (2002).
56. Musselman, E. D., Pelot, N. A. & Grill, W. M. Empirically Based Guidelines for Selecting Vagus Nerve Stimulation Parameters in Epilepsy and Heart Failure. Cold Spring Harbor perspectives in medicine (2018).
57. Yamamoto, T. Vagus nerve stimulation therapy: indications, programing, and outcomes. Neurologia medico-chirurgica 55, 407-415 (2015).
58. Clark, K. B., Krahl, S. E., Smith, D. C. & Jensen, R. A. Post-training unilateral vagal stimulation enhances retention performance in the rat. Neurobiology of learning and memory 63, 213-216 (1995).
59. Clark, K. B., et al. Posttraining electrical stimulation of vagal afferents with concomitant vagal efferent inactivation enhances memory storage processes in the rat. Neurobiology of learning and memory 70, 364-373 (1998).
60. Clark, K. B., Naritoku, D. K., Smith, D. C., Browning, R. A. & Jensen, R. A. Enhanced recognition memory following vagus nerve stimulation in human subjects. Nat Neurosci 2, 94-98 (1999).
61. Revesz, D., Tjernstrom, M., Ben-Menachem, E. & Thorlin, T. Effects of vagus nerve stimulation on rat hippocampal progenitor proliferation. Experimental neurology 214, 259-265 (2008).
62. Zuo, Y., Smith, D. C. & Jensen, R. A. Vagus nerve stimulation potentiates hippocampal LTP in freely-moving rats. Physiology & behavior 90, 583-589 (2007).
63. Rodenkirch, C., Liu, Y., Schriver, B. J. & Wang, Q. Locus coeruleus activation enhances thalamic feature selectivity via norepinephrine regulation of intrathalamic circuit dynamics. Nature Neuroscience 22, 120-133 (2019).
64. Schwartz, O., Pillow, J. W., Rust, N. C. & Simoncelli, E. P. Spike-triggered neural characterization. Journal of Vision 6, 484-507 (2006).
65. Hays, S. A., Rennaker, R. L. & Kilgard, M. P. Targeting Plasticity with Vagus Nerve Stimulation to Treat Neurological Disease. Progress in brain research 207, 275-299 (2013).
66. Ollerenshaw, Douglas R., Zheng, He J. V., Millard, Daniel C., Wang, Q. & Stanley, Garrett B. The Adaptive Trade-Off between Detection and Discrimination in Cortical Representations and Behavior. Neuron 81, 1152-1164 (2014).
67. Groves, D. A., Bowman, E. M. & Brown, V. J. Recordings from the rat locus coeruleus during acute vagal nerve stimulation in the anaesthetised rat. Neurosci Lett 379, 174-179 (2005).
68. Lopes, L. T., et al. Anatomical and functional connections between the locus coeruleus and the nucleus tractus solitarius in neonatal rats. Neuroscience 324, 446-468 (2016).
69. Goard, M. & Dan, Y. Basal forebrain activation enhances cortical coding of natural scenes. Nat Neurosci 12, 1444-1449 (2009).
70. Pinto, L., et al. Fast modulation of visual perception by basal forebrain cholinergic neurons. Nat Neurosci 16, 1857-1863 (2013).
71. McDevitt, Ross A., et al. Serotonergic versus Nonserotonergic Dorsal Raphe Projection Neurons: Differential Participation in Reward Circuitry. Cell reports 8, 1857-1869 (2014).
72. Samuels, E. R. & Szabadi, E. Functional neuroanatomy of the noradrenergic locus coeruleus: its roles in the regulation of arousal and autonomic function part I: principles of functional organisation. Current neuropharmacology 6, 235-253 (2008).
73. Dorr, A. E. & Debonnel, G. Effect of Vagus Nerve Stimulation on Serotonergic and Noradrenergic Transmission. Journal of Pharmacology and Experimental Therapeutics 318, 890 (2006).
74. Agnew, W. F., McCreery, D. B., Yuen, T. G. & Bullara, L. A. Histologic and physiologic evaluation of electrically stimulated peripheral nerve: considerations for the selection of parameters. Ann Biomed Eng 17, 39-60 (1989).
75. DeGiorgio, C., et al. Vagus nerve stimulation for epilepsy: randomized comparison of three stimulation paradigms. Neurology 65, 317-319 (2005).
76. Tehovnik, E. J., Slocum, W. M., Smirnakis, S. M. & Tolias, A. S. Microstimulation of visual cortex to restore vision. in Progress in Brain Research (ed. J. Verhaagen, et al.) 347-375 (Elsevier, 2009).
77. O/'Doherty, J. E., et al. Active tactile exploration using a brain-machine-brain interface. Nature 479, 228-231 (2011).
78. Kim, S., et al. Behavioral assessment of sensitivity to intracortical microstimulation of primate somatosensory cortex. Proc Natl Acad Sci USA 112, 15202-15207 (2015).
79. Bari, B. A., Ollerenshaw, D. R., Millard, D. C., Wang, Q. & Stanley, G. B. Behavioral and Electrophysiological Effects of Cortical Microstimulation Parameters. PLoS ONE 8, e82170 (2013).
80. Rodenkirch, C., Schriver, B. & Wang, Q. Brain-Machine Interfaces: Restoring and Establishing Communication Channels. in Neural Engineering (ed. L. Zhang & D. Kaplan) (Springer, 2016).
81. Romo, R., Hernandez, A., Zainos, A. & Salinas, E. Somatosensory discrimination based on cortical microstimulation. Nature 392, 387-390 (1998).
82. Panzeri, S., Safaai, H., De Feo, V. & Vato, A. Implications of the Dependence of Neuronal Activity on Neural Network States for the Design of Brain-Machine Interfaces. Frontiers in neuroscience 10, 165 (2016).
83. Schriver, B., Bagdasarov, S. & Wang, Q. Pupil-linked arousal modulates behavior in rats performing a whisker deflection direction discrimination task. Journal of Neurophysiology 120, 1655-1670 (2018).
84. Biesheuvel, J. D., Briaire, J. J., de Jong, M. A. M., Boehringer, S. & Frijns, J. H. M. Channel discrimination along all contacts of the cochlear implant electrode array and its relation to speech perception. International Journal of Audiology 58, 262-268 (2019).
85. Dinse, H. R., Ragert, P., Pleger, B., Schwenkreis, P. & Tegenthoff, M. Pharmacological modulation of perceptual learning and associated cortical reorganization. Science (New York, N. Y.) 301, 91-94 (2003).
86. Navarra, R. L., Clark, B. D., Gargiulo, A. T. & Waterhouse, B. D. Methylphenidate Enhances Early-Stage Sensory Processing and Rodent Performance of a Visual Signal Detection Task. Neuropsychopharmacology: official publication of the American College of Neuropsychopharmacology 42, 1326-1337 (2017).
87. Navarra, R. L., Clark, B. D., Zitnik, G. A. & Waterhouse, B. D. Methylphenidate and atomoxetine enhance sensory-evoked neuronal activity in the visual thalamus of male rats. Experimental and clinical psychopharmacology 21, 363-374 (2013).
88. Ben-Menachem, E., Revesz, D., Simon, B. J. & Silberstein, S. Surgically implanted and non-invasive vagus nerve stimulation: a review of efficacy, safety and tolerability. European journal of neurology 22, 1260-1268 (2015).
89. Weyand, T. G., et al. Burst and Tonic Response Modes in Thalamic Neurons During Sleep and Wakefulness Burst and Tonic Response Modes in Thalamic Neurons During Sleep and Wakefulness. 1107-1118 (2011).
90. Bezdudnaya, T., et al. Thalamic burst mode and inattention in the awake LGNd. Neuron 49, 421-432 (2006).
91. Sherman, S. M. A wake-up call from the thalamus. Nat Neurosci 4, 344-346 (2001).
92. Nicolelis, M. A. L. & Fanselow, E. E. Thalamocortical optimization of tactile processing according to behavioral state. Nature Neuroscience 5, 517-523 (2002).
93. Guillery, R. W., Feig, S. L. & Lozsádi, D. A. Paying attention to the thalamic reticular nucleus. Trends in Neurosciences 21, 28-32 (1998).
94. Ohara, P. T. & Lieberman, a.R. The thalamic reticular nucleus of the adult rat: experimental anatomical studies. Journal of neurocytology 14, 365-411 (1985).
95. Briggs, F., Mangun, G. R. & Usrey, W. M. Attention enhances synaptic efficacy and the signal-to-noise ratio in neural circuits. Nature 499, 476-480 (2013).
96. Holdefer, R. N. & Jacobs, B. L. Phasic stimulation of the locus coeruleus: effects on activity in the lateral geniculate nucleus. Experimental brain research 79, 444-452 (1994).
97. Stoelzel, C. R., Bereshpolova, Y. & Swadlow, H. A. Stability of Thalamocortical Synaptic Transmission across Awake Brain States. Journal of Neuroscience 29, 6851-6859 (2009).
98. Hirsch, J. A., Wang, X., Sommer, F. T. & Martinez, L. M. How Inhibitory Circuits in the Thalamus Serve Vision. Annual Review of Neuroscience 38, 309-329 (2015).
99. Livingstone, M. S. & Hubel, D. H. Effects of sleep and arousal on the processing of visual information in the cat. Nature 291, 554-561 (1981).
100. Wang, X., Sommer, F. T. & Hirsch, J.a. Inhibitory circuits for visual processing in thalamus. Current Opinion in Neurobiology 21, 726-733 (2011).
101. Pape, H.-C. & McCormick, D .A. Noradrenaline and serotonin selectively modulate thalamic burst firing by enhancing a hyperpolarization-activated cation current. Nature 340, 715-718 (1989).
102. Sara, Susan J. & Bouret, S. Orienting and Reorienting: The Locus Coeruleus Mediates Cognition through Arousal. Neuron 76, 130-141 (2012).
103. Berridge, C. W. & Waterhouse, B. D. The locus coeruleus—noradrenergic system: modulation of behavioral state and state-dependent cognitive processes. Brain Research Reviews 42, 33-84 (2003).
104. Avery, M. C. & Krichmar, J. L. Neuromodulatory Systems and Their Interactions: A Review of Models, Theories, and Experiments. Frontiers in neural circuits 11, 108-108 (2017).
105. Rios, M. U., et al. Protocol for Construction of Rat Nerve Stimulation Cuff Electrodes. Methods and protocols 2 (2019).
106. Paxinos, G. & Watson, C. The Rat Brain in Stereotaxic Coordinates (Academic Press, 1998).
107. Ben-Menachem, E. Vagus nerve stimulation, side effects, and long-term safety. J Clin Neurophysiol 18, 415-418 (2001).
108. Asconape, J. J., Moore, D. D., Zipes, D. P., Hartman, L. M. & Duffell, W. H., Jr. Bradycardia and asystole with the use of vagus nerve stimulation for the treatment of epilepsy: a rare complication of intraoperative device testing. Epilepsia 40, 1452-1454 (1999).
109. Chagas, A. M., et al. Functional analysis of ultra high information rates conveyed by rat vibrissal primary afferents. Front Neural Circuits 7, 190 (2013).
110. Adelman, T. L., Bialek, W. & Olberg, R. M. The information content of receptive fields. Neuron 40, 823-833 (2003).
111. Liu, Y., Rodenkirch, C., Moskowitz, N., Schriver, B. & Wang, Q. Dynamic Lateralization of Pupil Dilation Evoked by Locus Coeruleus Activation Results from Sympathetic, Not Parasympathetic, Contributions. Cell Reports 20, 3099-3112 (2017).
112. Mainen, Z. F. & Sejnowski, T. J. Reliability of spike timing in neocortical neurons. Science 268, 1503-1506 (1995).

1. A method of modifying a sensory processing in a subject, comprising applying a tonic vagus nerve stimulation to the subject wherein a modifying of sensory processing comprises increasing a sensory acuity of the subject.

2. The method of claim 1, wherein the sensory processing is modified within less than about 1 second.

3. The method of claim 1, wherein the modified sensory processing is transient, and wherein the effects of applying a tonic vagus nerve stimulation to the subject disappear within a minute of cessation of vagus nerve stimulation.

4. The method of claim 1, wherein the vagus nerve stimulation is continuous.

5. The method of claim 1, wherein the vagus nerve stimulation is discontinuous, and wherein a time period of a portion of the discontinuous stimulation wherein vagus nerve stimulation is not applied is not greater than about 7 to about 10 seconds.

6. The method of claim 1, wherein a rate of sensory related information transmitted by a thalamocortical relay neuron in a subject is increased by at least about 100 to 200% compared to a subject that does not receive the vagus nerve stimulation.

7. The method of claim 1, wherein the modifying of sensory processing comprises improving a sensory perception in a subject having one or more impaired senses.

8. The method of claim 7, wherein the subject has impaired senses caused by a condition selected from the group consisting of aging, traumatic brain injury (TBI), neurological disorders, fatigue, inattention, and neurodegeneration.

9. A method of modifying sensory processing in a subject, comprising:
detecting when the subject is in need of a sensory processing modification;
applying tonic vagus nerve stimulation to the subject to provide the sensory processing modification; and
discontinuing applying the sensory processing modification when the subject no longer is in need of the sensory processing modification.

10. The method of claim 9, wherein detecting that the subject is in need of the sensory processing modification comprises:
measuring a change in a signal from a first time to a second time;
determining a mean value and a variance value for the signal from the first time to the second time;
determining a measured value for the signal; and
applying tonic vagus nerve stimulation to the subject when the measured value is at least one to three standard deviations from the mean value.

11. The method of claim 10, wherein the signal being measured is selected from the group consisting of pupil diameter, EEG synchronization, relative power band strength, heart rate, heart rate variability, blood pressure, ECOG, respiratory rate, perspiration, skin conductivity, and signals recorded from invasive or noninvasive brain-machine interface.

12. The method of claim 9, wherein the vagus nerve stimulation is continuous.

13. The method of claim 9, wherein the vagus nerve stimulation is discontinuous , and wherein a time period of a portion of the discontinuous stimulation wherein vagus nerve stimulation is not applied is not greater than about 7 to about 10 seconds.

14. The method of claim 9, wherein a rate of sensory related information transmitted by a thalamocortical relay neuron in a subject is increased on average by at least about 100 to 200% compared to a subject that does not receive the vagus nerve stimulation.

15. The method of claim 14, wherein the modifying of sensory processing comprises improving a sensory perception in a subject having one or more impaired senses.

16. The method of claim 15, wherein the subject has impaired senses caused by a condition selected from the group consisting of aging, traumatic brain injury (TBI), neurological disorders, fatigue, inattention, and neurodegeneration.

17. A vagus nerve stimulation device adapted to apply a tonic vagus nerve stimulation to a subject to modify sensory processing in the subject, wherein a modifying of sensory processing comprises increasing a sensory acuity and wherein a time of applying the tonic vagus nerve stimulation is at least about 4 minutes.

18. The device of claim 17, wherein the modified sensory processing is transient, and wherein the effects of applying a tonic vagus nerve stimulation to the subject disappear within a minute of cessation of vagus nerve stimulation.

19. The device of claim 17, wherein the vagus nerve stimulation is continuous.

20. The device of claim 17, wherein the vagus nerve stimulation is discontinuous, and wherein a time period of a portion of the discontinuous stimulation wherein vagus nerve stimulation is not applied is not greater than about 7 to about 10 seconds.

21. The device of claim 17, wherein a rate of sensory related information transmitted by a thalamocortical relay neuron in a subject is increased on average by at least about 100 to 200% compared to a subject that does not receive the vagus nerve stimulation.

22. The device of claim 17, further comprising a prosthetic device adapted to be associated with a body part of the subject and wherein the prosthetic device is adapted to direct the vagus nerve stimulation to a cervical or auricular region of the subject.

23. The device of claim 22, wherein the prosthetic device is selected from the group consisting of eyeglasses, sunglasses, a hearing aid, a neck brace, a craniofacial prosthetic, a voice prosthetic, compression stimulation devices, sensory neuroprostheses, an orbital prostheses, a cervical collar, a halo vest, a dental implant, a facial implant, a helmet, a vehicle or machinery cockpit, machinery controls, a head-up display, a headset, a necklace, earrings, goggles, a tiara, a scarf, jewelry, a headdress, a headscarf, a hat, a tie, a bonnet, ear muffs, headphones, headsets, a shawl, a lanyard, a wig, a hood, a headband, a hair tie, a beret, a hair clip, a neck pillow, a shirt collar, a rifle scope, binoculars, a night vision device, a telescope, a virtual reality headset, a video game controller, a video game system, clothing, an adhesive patch, a blood pressure monitor, a heart rate monitor, an oximeter, a watch, a smart watch, a phone, and 3D glasses.
